(19) United States
(12) Patent Application Publication
(10) Pub. No.: US 2017/0209616 A1
(43) Pub. Date: Jul. 27, 2017

(54) ABSORBENT CORES WITH HIGH MOLECULAR WEIGHT SUPERABSORBENT IMMOBILIZER

(71) Applicant: The Procter & Gamble Company, Cincinnati, OH (US)

(72) Inventor: Robert Haines TURNER, Cincinnati, OH (US)

(21) Appl. No.: 15/413,723

(22) Filed: Jan. 24, 2017

Related U.S. Application Data
(60) Provisional application No. 62/287,090, filed on Jan. 26, 2016.
(60) Provisional application No. 62/312,479, filed on Mar. 24, 2016.

(51) Int. Cl. A61L 15/60; A61L 15/24

(52) U.S. Cl. CPC A61L 15/60; A61L 15/24; A61F 13/534

(57) ABSTRACT

An absorbent article comprising an absorbent core, the absorbent core comprising superabsorbent polymer material and a superabsorbent immobilizer comprising at least about 50% of one or more polymers having a peak molecular weight of at least about 10 kg/mol.

### FIELD OF THE INVENTION

The present invention generally relates to an absorbent core for use in an absorbent article, and more particularly to an absorbent core with a superabsorbent immobilizer comprising high molecular weight polymers.

### BACKGROUND OF THE INVENTION

Disposable absorbent articles for receiving and retaining bodily discharges such as urine or feces are generally known in the art. Examples of these include disposable diapers, training pants and adult incontinence articles. Typically, disposable diapers comprise a liquid pervious topsheet that faces the wearer's body, a liquid impervious backsheet that faces the wearer's clothing and an absorbent core interposed between the liquid pervious topsheet and the backsheet.

An important component of disposable absorbent articles is the absorbent core structure. The absorbent core structure typically includes absorbent polymer material, such as hydrogel-forming polymer material, also referred to as absorbent gelling material, AGM, or super-absorbent polymer, SAP. This absorbent polymer material ensures that large amounts of bodily fluids, e.g. urine, can be absorbed by the absorbent article during its use and be locked away, thus providing low rewet and good skin dryness.

Traditionally, the absorbent polymer material is incorporated into the absorbent core structure with cellulose or cellulosic fibres. Now, thinner absorbent core structures can be made by the reduction or elimination of these cellulose fibres from the absorbent core structures, whereby the core structures can still acquire and store large quantities of discharged body fluids, in particular urine. To maintain the mechanical stability of these absorbent core structures, a superabsorbent immobilizer (SI) may be added to stabilize the absorbent polymer material. In some cases, this SI may be a fiberized structure. The fiberized structure may be an adhesive, that is, a material that has a base polymer, along with other materials such as tackifiers, plasticizers, oils, and/or waxes, for example. However, these additive materials in the fiberized adhesive, other than the base polymer, can migrate during product use and create instability issues that negatively affect the performance and consumer impression of the article and create the need for more adhesive to be used to maintain function. Thus, the use of a fiberized adhesive in the core adds to the cost of the absorbent article and creates stability issues that must be managed. Therefore, there is a need to reduce or eliminate the additive materials in the composition that is used to immobilize the superabsorbent polymer material. Accordingly, there is a need for an immobilizer for the superabsorbent polymer that is less expensive and that can provide improved stability to the absorbent core.

### SUMMARY OF THE INVENTION

An absorbent article comprising an absorbent core, wherein the absorbent core comprises superabsorbent polymer material and a superabsorbent immobilizer. The superabsorbent immobilizer comprises at least one polymer having a peak molecular weight of at least about 10 kg/mol, as determined using the gel permeation chromatography method described herein, and the combined polymers each having a peak molecular weight of at least about 10 kg/mol comprise at least about 50% by weight of the superabsorbent immobilizer.

### BRIEF DESCRIPTION OF DRAWINGS

FIG. 1 is a plan view of a diaper in accordance with an embodiment of the present invention.

FIG. 2 is a cross sectional view of the diaper shown in FIG. 1 taken along the sectional line 2-2 of FIG. 1.

FIG. 3 is a partial cross sectional view of an absorbent core layer in accordance with an embodiment of this invention.

FIG. 4 is a partial cross sectional view of an absorbent core layer in accordance with another embodiment of this invention.

FIG. 5 is a plan view of the absorbent core layer illustrated in FIG. 3.

FIG. 6 is a plan view of a second absorbent core layer in accordance with an embodiment of this invention.

FIG. 7A is a partial sectional view of an absorbent core comprising a combination of the first and second absorbent core layers illustrated in FIGS. 5 and 6.

FIG. 7B is a partial sectional view of an absorbent core comprising a combination of the first and second absorbent core layers illustrated in FIGS. 5 and 6.

FIG. 8 is a plan view of the absorbent core illustrated in FIGS. 7a and 7b.

FIG. 9 is a schematic illustration of a process for making an absorbent core in accordance with an embodiment of the present invention.

FIG. 10 is a top view of an absorbent core according to the invention with some of the layers partially removed.

FIG. 11 is a transversal cross-section of the core of FIG. 10 along 2-2.

FIG. 12 shows a transversal cross-section of the article of FIG. 10 along 2-2 when the absorbent core has swollen after absorbing a fluid.

FIG. 13 is the longitudinal cross-section of the core of FIG. 10 along 3-3, showing an optional dual layer construction for the absorbent layer;

FIG. 14 is a close-up view of a section of FIG. 13.

### DETAILED DESCRIPTION

### Definitions

“Absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” or “absorbent structure” means a structure typically disposed between a topsheet and backsheet of an absorbent article for absorbing and containing liquid received by the absorbent article and may comprise one or more substrates, absorbent polymer material disposed on the one or more substrates, and a thermoplastic composition, such as a superabsorbent immobilizer, on the absorbent particulate polymer material and at least a portion of the one or more substrates for immobilizing the absorbent particulate polymer material on the one or more substrates. In a multilayer absorbent core, the absorbent core may also include a cover layer. The one or more substrates and the cover layer may comprise a nonwoven. Further, the absorbent core may be substantially cellulose free. The absorbent core does not include an acquisition system, a topsheet, or a backsheet of the absorbent article. In a certain embodiment, the absorbent core may consist essentially of the one or more substrates, the absorbent polymer material, a superabsorbent immobilizer that may be a fiberized structure, and optionally the cover layer.

“Absorbent particulate polymer material” is used herein to refer to an absorbent polymer material which is in particulate form so as to be flowable in the dry state.

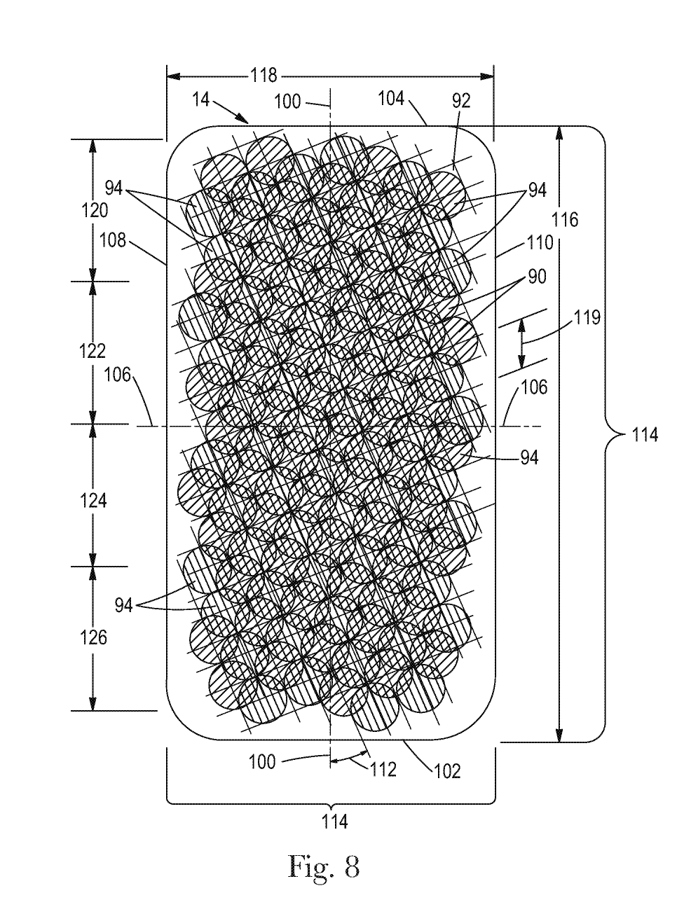

“Absorbent particulate polymer material area” as used herein refers to the area of the core wherein the first substrate and second substrate are separated by a multiplicity of superabsorbent particles. In FIG. 8, the boundary of the absorbent particulate polymer material area is defined by the perimeter of the overlapping circles. There may be some extraneous superabsorbent particles outside of this perimeter between the first substrate and second substrate.

“Airfelt” is used herein to refer to comminuted wood pulp, which is a form of cellulosic fiber.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Fiber” and “filament” are used interchangeably.

“Fiberized structure” as used herein is understood to comprise a polymer composition from which strands or a net structure is formed and applied to the superabsorbent material with the intent to immobilize the superabsorbent material in both the dry and wet state. The fiberized structure of the present invention forms a fibrous network over, around, and/or between the superabsorbent material.

A “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”.

As used herein, the term “substantially” means generally the same or uniform but allowing for or having minor fluctuations from a defined property, definition, etc. For example, small measurable or immeasurable fluctuations in a measured property described herein, such as viscosity, melting point, etc. may result from human error or methodology precision. Other fluctuations are caused by inherent variations in the manufacturing process, thermal history of a formulation, and the like. The compositions of the present invention, nonetheless, would be said to be substantially having the property as reported.

“Substantially cellulose free” is used herein to describe an article, such as an absorbent core, that contains less than 10% by weight cellulosic fibers, less than 5% cellulosic fibers, less than 1% cellulosic fibers, no cellulosic fibers, or no more than an immaterial amount of cellulosic fibers. An immaterial amount of cellulosic material would not materially affect the thinness, flexibility, or absorbency of an absorbent core.

As used herein, the term “substrate” means any item having at least a partially or fully solidified fiber or planar surface. In some cases, a single substrate may be positioned in a way that it is referred to as two or more substrates; for example a folded film or folded non-woven, or two sides of a cardboard sheet folded over, wherein the two sides are adhesively bonded together. The substrates can be impermeable, permeable, porous or nonporous.

“Superabsorbent polymer material”, “absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01).

“Superabsorbent immobilizer” or “SI” means a composition that is applied to the superabsorbent material with the intent to immobilize the superabsorbent material in both the dry and wet state. The SI may be a fiberized structure with, for example, microfibers or nanofibers, or may be a film, discrete blobs of material, or some other form.

### Article

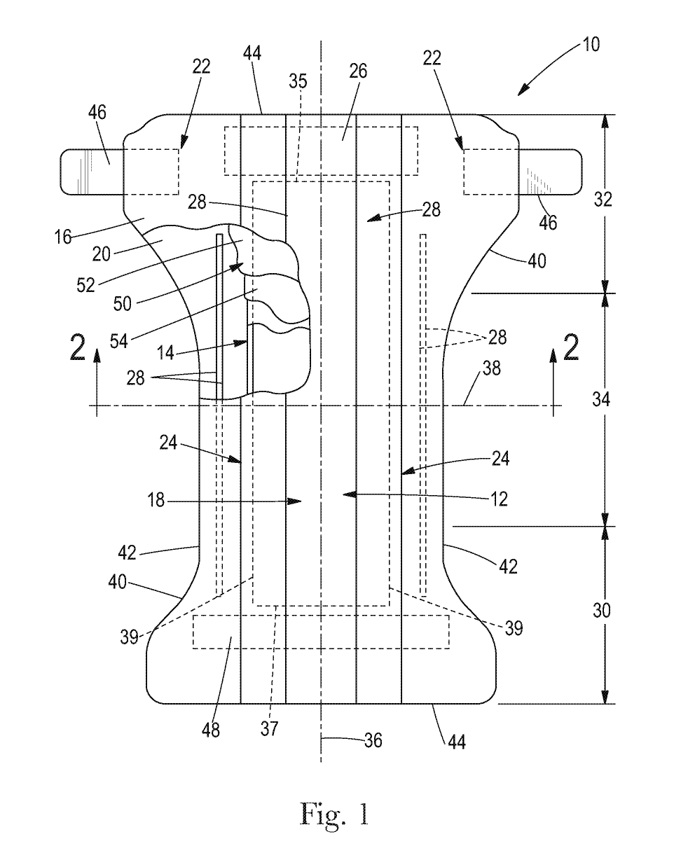

FIG. 1 is a plan view of an article, such as a diaper, 10 according to a certain embodiment of the present invention. The diaper 10 is shown in its flat out, uncontracted state (i.e., without elastic induced contraction) and portions of the diaper 10 are cut away to more clearly show the underlying structure of the diaper 10. A portion of the diaper 10 that contacts a wearer is facing the viewer in FIG. 1. The diaper 10 generally may comprise a chassis 12 and an absorbent core 14 disposed in the chassis.

The chassis 12 of the diaper 10 in FIG. 1 may comprise the main body of the diaper 10. The chassis 12 may comprise an outer covering 16 including a topsheet 18, which may be liquid pervious, and/or a backsheet 20, which may be liquid impervious. The absorbent core 14 may be encased between the topsheet 18 and the backsheet 20. The chassis 12 may also include side panels 22, elasticized leg cuffs 24, and an elastic waist feature 26.

The leg cuffs 24 and the elastic waist feature 26 may each typically comprise elastic members 28. One end portion of the diaper 10 may be configured as a first waist region 30 of the diaper 10. An opposite end portion of the diaper 10 may be configured as a second waist region 32 of the diaper 10. An intermediate portion of the diaper 10 may be configured as a crotch region 34, which extends longitudinally between the first and second waist regions 30 and 32. The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 is depicted in FIG. 1 with its longitudinal axis 36 and its transverse axis 38. The periphery 40 of the diaper 10 is defined by the outer edges of the diaper 10 in which the longitudinal edges 42 run generally parallel to the longitudinal axis 36 of the diaper 10 and the end edges 44 run between the longitudinal edges 42 generally parallel to the transverse axis 38 of the diaper 10. The chassis 12 may also comprise a fastening system, which may include at least one fastening member 46 and at least one stored landing zone 48.

The diaper 10 may also include such other features as are known in the art including front and rear ear panels, waist cap features, elastics and the like to provide better fit, containment and aesthetic characteristics. Such additional features are well known in the art and are e.g., described in U.S. Pat. No. 3,860,003 and U.S. Pat. No. 5,151,092.

In order to keep the diaper 10 in place about the wearer, at least a portion of the first waist region 30 may be attached by the fastening member 46 to at least a portion of the second waist region 32 to form leg opening(s) and an article waist. When fastened, the fastening system carries a tensile load around the article waist. The fastening system may allow an article user to hold one element of the fastening system, such as the fastening member 46, and connect the first waist region 30 to the second waist region 32 in at least two places. This may be achieved through manipulation of bond strengths between the fastening device elements.

According to certain embodiments, the diaper 10 may be provided with a re-closable fastening system or may alternatively be provided in the form of a pant-type diaper. When the absorbent article is a diaper, it may comprise a re-closable fastening system joined to the chassis for securing the diaper to a wearer. When the absorbent article is a pant-type diaper, the article may comprise at least two side panels joined to the chassis and to each other to form a pant. The fastening system and any component thereof may include any material suitable for such a use, including but not limited to plastics, films, foams, nonwoven, woven, paper, laminates, fiber reinforced plastics and the like, or combinations thereof. In certain embodiments, the materials making up the fastening device may be flexible. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the diaper 10 with other features added to form the composite diaper structure. While the topsheet 18, the backsheet 20, and the absorbent core 14 may be assembled in a variety of well-known configurations, preferred diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

The topsheet 18 in FIG. 1 may be fully or partially elasticized or may be foreshortened to provide a void space between the topsheet 18 and the absorbent core 14. Exemplary structures including elasticized or foreshortened topsheets are described in more detail in U.S. Pat. No. 5,037,416 and U.S. Pat. No. 5,269,775.

The topsheet may be compliant, soft feeling, and non-irritating to the wearer's skin and may be elastically stretchable in one or more directions. Further, the topsheet may be liquid pervious, permitting liquids (e.g., menses, urine, and/or runny feces) to penetrate through its thickness. Various topsheets may also comprise a hydrophilic material, for example, which is configured to draw bodily fluids into an absorbent core of the chassis when these fluids are expelled from the body. A suitable topsheet may be manufactured from a wide range of materials, such as woven and nonwoven materials, apertured or hydroformed thermoplastic films, apertured nonwovens, porous foams, reticulated foams, reticulated thermoplastic films, and/or thermoplastic scrims, for example. Suitable apertured films may comprise those described in U.S. Pat. Nos. 3,929,135, 4,324,246, 4,342,314, 4,463,045, 5,006,394, 5,628,097, 5,916,661, 6,545,197, and 6,107,539.

Apertured film or nonwoven topsheets typically may be pervious to bodily exudates, yet non-absorbent, and have a reduced tendency to allow fluids to pass back through and rewet the wearer's skin. Suitable woven and nonwoven materials may comprise natural fibers, such as, for example, wood or cotton fibers, synthetic fibers, such as, for example, polyester, polypropylene, or polyethylene fibers, or combinations thereof. If the topsheet comprises fibers, the fibers may be spunbond, carded, wet-laid, meltblown, hydroentangled, or otherwise processed, for example, as is generally known in the art.

The topsheet may comprise a skin care lotion. Examples of suitable lotions include, but are not limited to, those described in U.S. Pat. Nos. 5,607,760; 5,609,587; 5,635,191; 5,643,588; and 5,968,025, and as described in U.S. Application No. 61/391,353, and as described in U.S. Pub. No. 2014-0257216. Beyond these compositions, the absorbent article may comprise soluble cyclodextrin derivatives such as those described in U.S. Pub. No. 2014/0274870.

Additionally, the topsheet of the present disclosure may be a tufted laminate web as disclosed in U.S. Pat. No. 7,410,683, and/or may be an apertured web as disclosed in PCT/CN2014/083769 having an international filing date of Aug. 6, 2014.

In one embodiment, the topsheet may comprise graphics such that depth perception is created as described in U.S. Pat. No. 7,163,528. In other embodiments, the topsheet may be an integrated acquisition layer and topsheet as described in U.S. Ser. No. 14/680,426 or 14/634,928.

In one embodiment, the absorbent article may comprise a backsheet. The backsheet may be impervious, or at least partially impervious, to fluids or body exudates (e.g., menses, urine, and/or runny feces) and may be manufactured from a thin plastic film, although other flexible liquid impervious materials may also be used. The backsheet may prevent the body exudates or fluids absorbed and contained in an absorbent core of the absorbent article from wetting articles which contact the absorbent article, such as bedsheets, pajamas, clothes, and/or undergarments. The backsheet may comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). A suitable backsheet may comprise a polyethylene film having a thickness of from about 0.012 mm (0.5 mils) to about 0.051 mm (2.0 mils). Examples of polyethylene films are manufactured by Clopay Corporation of Cincinnati, Ohio, under the designation BR-120 and BR-121, and by Tredegar Film Products of Terre Haute, Ind., under the designation XP-39385.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m2 to about 35 g/m2. The backsheet can be typically positioned adjacent the outer-facing surface of the absorbent core and can be joined thereto. For example, the backsheet may be secured to the absorbent core by a uniform continuous layer of adhesive, a patterned layer of adhesive, or an array of separate lines, spirals, or spots of adhesive. Illustrative, but non-limiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986. Another suitable attachment device including several lines of adhesive filaments swirled into a spiral pattern is illustrated by the apparatus and methods shown in U.S. Pat. Nos. 3,911,173; 4,785,996; and 4,842,666. Alternatively, the attachment device may include heat bonds, pressure bonds, ultrasonic bonds, dynamic mechanical bonds, or any other suitable attachment device or combinations of these attachment devices.

In one embodiment, the backsheet may be embossed and/or matte-finished to provide a more cloth-like appearance. Further, the backsheet may permit vapors to escape from the absorbent core of the absorbent article (i.e., the backsheet is breathable) while still preventing, or at least inhibiting, fluids or body exudates from passing through the backsheet. In one embodiment, the size of the backsheet may be dictated by the size of the absorbent article and the design or configuration of the absorbent article to be formed, for example.

The backsheet 20 may be joined with the topsheet 18. Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper 10 while still preventing liquid exudates from passing through the backsheet 10. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont. Other breathable backsheets including nonwoven webs and apertured formed films are described in U.S. Pat. No. 5,571,096 issued to Dobrin et al. on Nov. 5, 1996.

In certain embodiments, the backsheet of the present invention may have a water vapor transmission rate (WVTR) of greater than about 2000 g/24 h/m², greater than about 3000 g/24 h/m², greater than about 5000 g/24 h/m², greater than about 6000 g/24 h/m², greater than about 7000 g/24 h/m², greater than about 8000 g/24 h/m², greater than about 9000 g/24 h/m², greater than about 10000 g/24 h/m², greater than about 11000 g/24 h/m², greater than about 12000 g/24 h/m², greater than about 15000 g/24 h/m², measured according to WSP 70.5 (08) at 37.8° C. and 60% Relative Humidity.

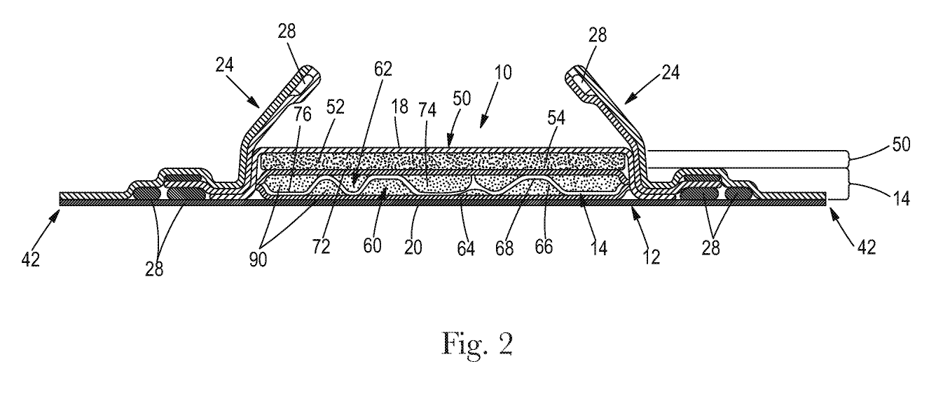

FIG. 2 shows a cross section of FIG. 1 taken along the sectional line 2-2 of FIG. 1. Starting from the wearer facing side, the diaper 10 may comprise the topsheet 18, the components of the absorbent core 14, and the backsheet 20. According to a certain embodiment, the diaper 10 may also comprise an acquisition system 50 disposed between the liquid permeable topsheet 18 and a wearer facing side of the absorbent core 14. The acquisition system 50 may be in direct contact with the absorbent core. The acquisition system 50 may comprise a single layer or multiple layers, such as an upper acquisition layer 52 facing towards the wearer's skin and a lower acquisition 54 layer facing the garment of the wearer. According to a certain embodiment, the acquisition system 50 may function to receive a surge of liquid, such as a gush of urine. In other words, the acquisition system 50 may serve as a temporary reservoir for liquid until the absorbent core 14 can absorb the liquid.

In a certain embodiment, the acquisition system 50 may comprise chemically cross-linked cellulosic fibers. Such cross-linked cellulosic fibers may have desirable absorbency properties. Exemplary chemically cross-linked cellulosic fibers are disclosed in U.S. Pat. No. 5,137,537. According to certain embodiments, the cross-linked cellulosic fibers may be crimped, twisted, or curled, or a combination thereof including crimped, twisted, and curled.

In a certain embodiment, one or both of the upper and lower acquisition layers 52 and 54 may comprise a non-woven, which may be hydrophilic. Further, according to a certain embodiment, one or both of the upper and lower acquisition layers 52 and 54 may comprise the chemically cross-linked cellulosic fibers, which may or may not form part of a nonwoven material. According to an exemplary embodiment, the upper acquisition layer 52 may comprise a nonwoven, without the cross-linked cellulosic fibers, and the lower acquisition layer 54 may comprise the chemically cross-linked cellulosic fibers. Further, according to an embodiment, the lower acquisition layer 54 may comprise the chemically cross-linked cellulosic fibers mixed with other fibers such as natural or synthetic polymeric fibers. According to exemplary embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof. According to a particular embodiment, the lower acquisition layer 54 has a total dry weight, the cross-linked cellulosic fibers are present on a dry weight basis in the upper acquisition layer in an amount from about 30% to about 95% by weight of the lower acquisition layer 54, and the other natural or synthetic polymeric fibers are present on a dry weight basis in the lower acquisition layer 54 in an amount from about 70% to about 5% by weight of the lower acquisition layer 54.

According to a certain embodiment, the lower acquisition layer 54 desirably has a high fluid uptake capability. Fluid uptake is measured in grams of absorbed fluid per gram of absorbent material and is expressed by the value of “maximum uptake.” A high fluid uptake corresponds therefore to a high capacity of the material and is beneficial, because it ensures the complete acquisition of fluids to be absorbed by an acquisition material. According to exemplary embodiments, the lower acquisition layer 54 has a maximum uptake of about 10 g/g.

Suitable non-woven materials for the upper and lower acquisition layers 52 and 54 include, but are not limited to SMS material, comprising a spunbonded, a melt-blown and a further spunbonded layer. In certain embodiments, permanently hydrophilic non-wovens, and in particular, nonwovens with durably hydrophilic coatings are desirable. Additional suitable embodiments may in particular be formed by a nonwoven web, such as a carded nonwoven, a spunbond nonwoven (“S”) or a meltblown nonwoven (“M”), and laminates of any of these. For example spunmelt polypropylene nonwovens are suitable, in particular those having a laminate web SMS, or SMMS, or SSMMS, structure, and having a basis weight range of about 5 gsm to 15 gsm. Suitable materials are for example disclosed in U.S. Pat. No. 7,744,576, US2011/0268932A1, US2011/0319848A1, or US2011/0250413A1. Nonwoven materials provided from synthetic fibers may be used, such as polyethylene, polyethylene terephthalate, and in particular polypropylene.

As polymers used for nonwoven production may be inherently hydrophobic, they may be coated with hydrophilic coatings. One way to produce nonwovens with durably hydrophilic coatings, is via applying a hydrophilic monomer and a radical polymerization initiator onto the nonwoven, and conducting a polymerization activated via UV light resulting in monomer chemically bound to the surface of the nonwoven as described in co-pending U.S. Patent Publication No. 2005/0159720. Another way to produce nonwovens with durably hydrophilic coatings is to coat the nonwoven with hydrophilic nanoparticles as described in co-pending applications U.S. Pat. No. 7,112,621 to Rohrbaugh et al. and in PCT Application Publication WO 02/064877.

Typically, nanoparticles have a largest dimension of below 750 nm. Nanoparticles with sizes ranging from 2 to 750 nm may be economically produced. An advantage of nanoparticles is that many of them can be easily dispersed in water solution to enable coating application onto the nonwoven, they typically form transparent coatings, and the coatings applied from water solutions are typically sufficiently durable to exposure to water. Nanoparticles can be organic or inorganic, synthetic or natural. Inorganic nanoparticles generally exist as oxides, silicates, and/or, carbonates. Typical examples of suitable nanoparticles are layered clay minerals (e.g., LAPONITE™ from Southern Clay Products, Inc. (USA), and Boehmite alumina (e.g., Disperal P2™ from North American Sasol. Inc.). According to a certain embodiment, a suitable nanoparticle coated non-woven is that disclosed in patent application Ser. No. 10/758,066 entitled “Disposable absorbent article comprising a durable hydrophilic core wrap” to Ekaterina Anatolyevna Ponomarenko and Mattias NMN Schmidt.

Further useful non-wovens are described in U.S. Pat. No. 6,645,569 to Cramer et al., U.S. Pat. No. 6,863,933 to Cramer et al., U.S. Pat. No. 7,112,621 to Rohrbaugh et al., and co-pending patent application Ser. No. 10/338,603 to Cramer et al. and Ser. No. 10/338,610 to Cramer et al.

In some cases, the nonwoven surface can be pre-treated with high energy treatment (corona, plasma) prior to application of nanoparticle coatings. High energy pre-treatment typically temporarily increases the surface energy of a low surface energy surface (such as PP) and thus enables better wetting of a nonwoven by the nanoparticle dispersion in water.

Notably, permanently hydrophilic non-wovens are also useful in other parts of an absorbent article. For example, topsheets and absorbent core layers comprising permanently hydrophilic non-wovens as described above have been found to work well.

According to a certain embodiment, the upper acquisition layer 52 may comprise a material that provides good recovery when external pressure is applied and removed. Further, according to a certain embodiment, the upper acquisition layer 52 may comprise a blend of different fibers selected, for example from the types of polymeric fibers described above. In some embodiments, at least a portion of the fibers may exhibit a spiral-crimp which has a helical shape. In some embodiments, the upper acquisition layer 52 may comprise fibers having different degrees or types of crimping, or both. For example, one embodiment may include a mixture of fibers having about 8 to about 12 crimps per inch (cpi) or about 9 to about 10 cpi, and other fibers having about 4 to about 8 cpi or about 5 to about 7 cpi. Different types of crimps include, but are not limited to a 2D crimp or “flat crimp” and a 3D or spiral-crimp. According to a certain embodiment, the fibers may include bi-component fibers, which are individual fibers each comprising different materials, usually a first and a second polymeric material. It is believed that the use of side-by-side bi-component fibers is beneficial for imparting a spiral-crimp to the fibers.

The upper acquisition layer 52 may be stabilized by a latex binder, for example a styrene-butadiene latex binder (SB latex), in a certain embodiment. Processes for obtaining such lattices are known, for example, from EP 149 880 (Kwok) and US 2003/0105190 (Diehl et al.). In certain embodiments, the binder may be present in the upper acquisition layer 52 in excess of about 12%, about 14% or about 16% by weight. For certain embodiments, SB latex is available under the trade name GENFLO™ 3160 (OMNOVA Solutions Inc.; Akron, Ohio).

### Absorbent Core

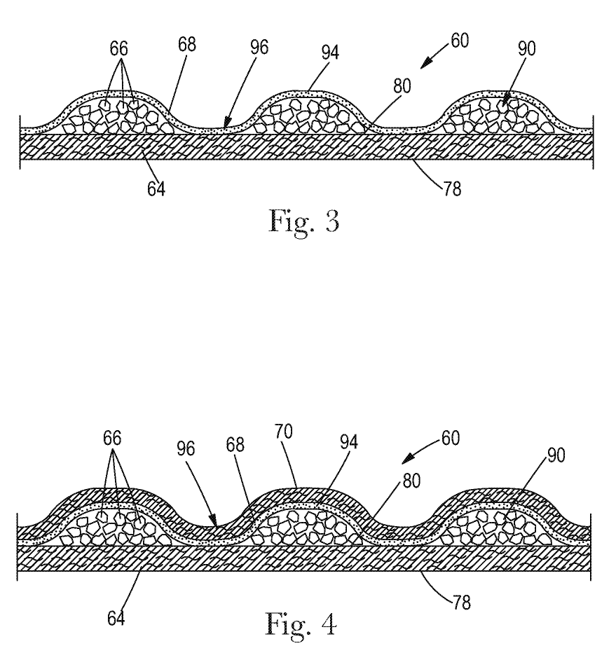

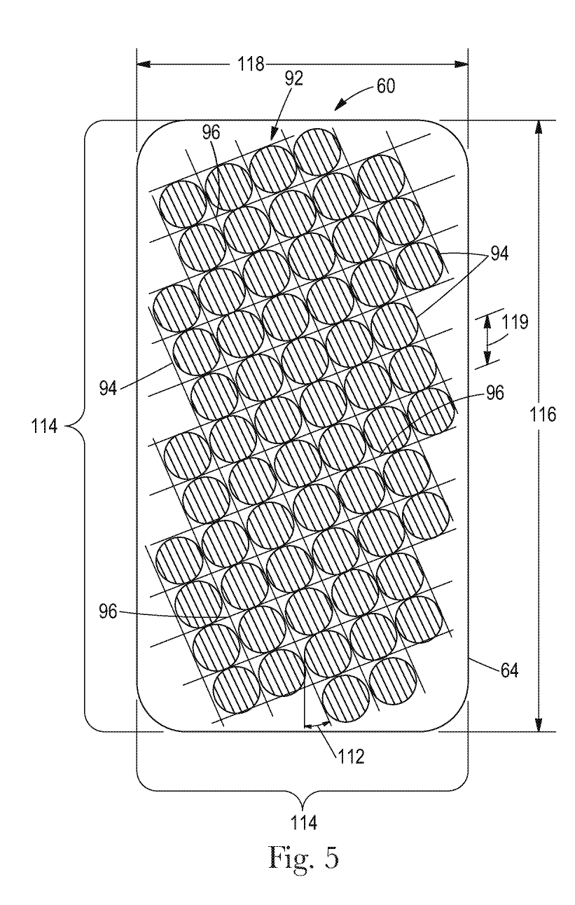

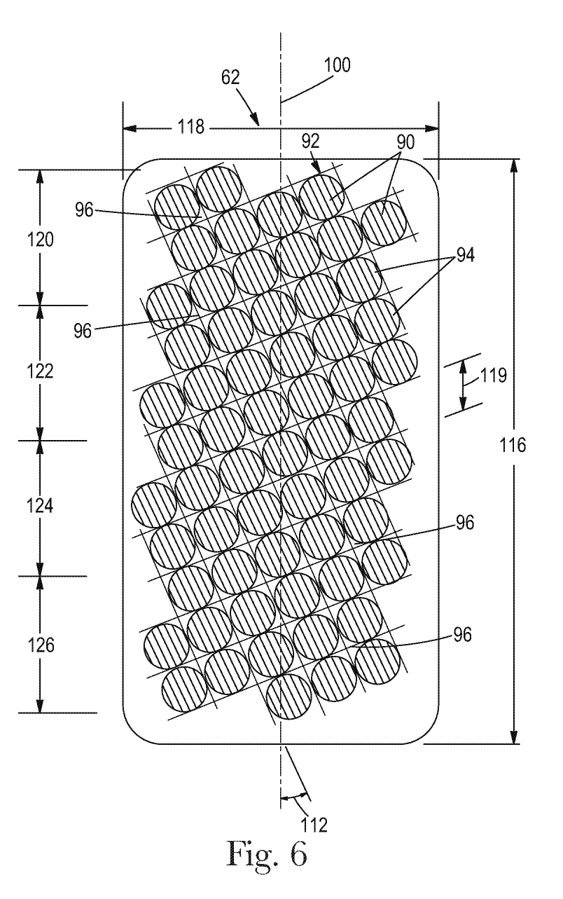

The absorbent core 14 in FIGS. 1-8 generally is disposed between the topsheet 18 and the backsheet 20 and may comprise two layers, a first absorbent layer 60 and a second absorbent layer 62. As best shown in FIG. 3, the first absorbent layer 60 of the absorbent core 14 comprises a substrate 64, an absorbent particulate polymer material (such as a superabsorbent polymer material) 66 deposited on the substrate 64, and a superabsorbent immobilizer, (SI), 68 on the absorbent particulate polymer material 66 and at least portions of the first substrate 64 as a means for covering and immobilizing the absorbent particulate polymer material 66 on the first substrate 64. The superabsorbent immobilizer may be a thermoplastic composition and/or may be a fiberized structure. According to another embodiment illustrated in FIG. 4, the first absorbent layer 60 of the absorbent core 14 may also include a cover layer 70 on the SI 68.

Likewise, as best illustrated in FIG. 2, the second absorbent layer 62 of the absorbent core 14 may also include a substrate 72, an absorbent particulate polymer material (such as a superabsorbent polymer material) 74 on the second substrate 72, and a SI that may be a thermoplastic composition and/or a fiberized structure 76 on the absorbent particulate polymer material 74 and at least a portion of the second substrate 72 for immobilizing the absorbent particulate polymer material 74 on the second substrate 72. Although not illustrated, the second absorbent layer 62 may also include a cover layer such as the cover layer 70 illustrated in FIG. 4. The first and second absorbent layers may be combined together such that at least a portion of the superabsorbent immobilizer of the first absorbent layer contacts at least a portion of the superabsorbent immobilizer of the second absorbent layer.

The substrate 64 of the first absorbent layer 60 may be a dusting layer, in other embodiments, a core cover, and has a first surface or outer surface 78 which faces the backsheet 20 of the diaper 10 and a second surface or inner surface 80 which faces the absorbent particulate polymer material 66. Likewise, the substrate 72 of the second absorbent layer 62 may be referred to as a core cover and has a first surface or outer surface 82 facing the topsheet 18 of the diaper 10 and a second surface or inner surface 84 facing the absorbent particulate polymer material 74. In some embodiments, the first substrate 64 and the second substrate 72 may both be core covers or core wrap material. The first and second substrates 64 and 72 may be adhered to one another with adhesive about the periphery to form an envelope about the absorbent particulate polymer materials 66 and 74 to hold the absorbent particulate polymer material 66 and 74 within the absorbent core 14. The absorbent core may then have a front edge 35, a back edge 37, and two side edges 39. The bonded periphery at the front edge 35 may form a front end seal and the bonded periphery at the back edge may form a back end seal.

According to a certain embodiment, the substrates 64 and 72 of the first and second absorbent layers 60 and 62 may be a nonwoven material, such as those nonwoven materials described above. In certain embodiments, the nonwovens are porous and in one embodiment has a pore size of about 32 microns.

As illustrated in FIGS. 1-8, the absorbent particulate polymer material 66 and 74 is deposited on the respective substrates 64 and 72 of the first and second absorbent layers 60 and 62 in clusters 90 of particles to form a grid pattern 92 comprising land areas 94 and junction areas 96 between the land areas 94. As defined herein, land areas 94 are areas where the SI does not contact the nonwoven substrate or the auxiliary adhesive (discussed below) directly; junction areas 96 are areas where the SI does contact the nonwoven substrate or the auxiliary adhesive directly. The junction areas 96 in the grid pattern 92 contain little or no absorbent particulate polymer material 66 and 74. The land areas 94 and junction areas 96 can have a variety of shapes including, but not limited to, circular, oval, square, rectangular, triangular, and the like.

The grid pattern shown in FIG. 8 is a square grid with regular spacing and size of the land areas. Other grid patterns including hexagonal, rhombic, orthorhombic, parallelogram, triangular, rectangular, and combinations thereof may also be used. The spacing between the grid lines may be regular or irregular.

The size of the land areas 94 in the grid patterns 92 may vary. According to certain embodiments, the width 119 of the land areas 94 in the grid patterns 92 ranges from about 8 mm to about 12 mm. In a certain embodiment, the width of the land areas 94 is about 10 mm. The junction areas 96, on the other hand, in certain embodiments, have a width or larger span of less than about 5 mm, less than about 3 mm, less than about 2 mm, less than about 1.5 mm, less than about 1 mm, or less than about 0.5 mm.

As shown in FIG. 8, the absorbent core 14 has a longitudinal axis 100 extending from a rear end 102 to a front end 104 and a transverse axis 106 perpendicular to the longitudinal axis 100 extending from a first edge 108 to a second edge 110. The grid pattern 92 of absorbent particulate polymer material clusters 90 is arranged on the substrates 64 and 72 of the respective absorbent layers 60 and 62 such that the grid pattern 92 formed by the arrangement of land areas 94 and junction areas 96 forms a pattern angle 112. The pattern angle 112 may be 0, greater than 0, or 15 to 30 degrees, or from about 5 to about 85 degrees, or from about 10 to about 60 degrees, or from about 15 to about 30 degrees.

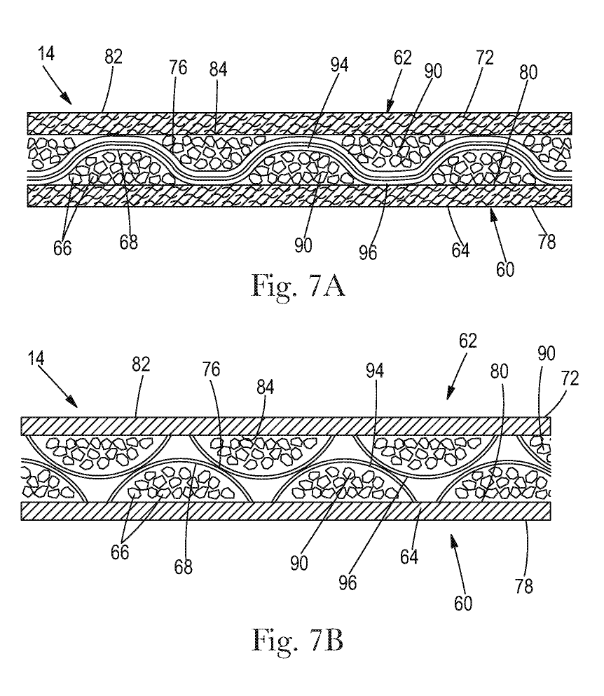

As best seen in FIGS. 7a, 7b, and 8, the first and second layers 60 and 62 may be combined to form the absorbent core 14. The absorbent core 14 has a superabsorbent polymer material area (or absorbent particulate area) 114 bounded by a pattern length 116 and a pattern width 118. The extent and shape of the superabsorbent polymer material area 114 may vary depending on the desired application of the absorbent core 14 and the particular absorbent article in which it may be incorporated. In a certain embodiment, however, the superabsorbent polymer material area 114 extends substantially entirely across the absorbent core 14, such as is illustrated in FIG. 8.

The first and second absorbent layers 60 and 62 may be combined together to form the absorbent core 14 such that the grid patterns 92 of the respective first and second absorbent layers 62 and 64 are offset from one another along the length and/or width of the absorbent core 14. The respective grid patterns 92 may be offset such that the superabsorbent polymer material 66 and 74 is substantially continuously distributed across the superabsorbent polymer area 114. In a certain embodiment, superabsorbent polymer material (or absorbent particulate polymer material) 66 and 74 is substantially continuously distributed across the absorbent particulate polymer material area 114 despite the individual grid patterns 92 comprising absorbent particulate polymer material 66 and 74 discontinuously distributed across the first and second substrates 64 and 72 in clusters 90. In a certain embodiment, the grid patterns may be offset such that the land areas 94 of the first absorbent layer 60 face the junction areas 96 of the second absorbent layer 62 and the land areas of the second absorbent layer 62 face the junction areas 96 of the first absorbent layer 60. When the land areas 94 and junction areas 96 are appropriately sized and arranged, the resulting combination of absorbent particulate polymer material 66 and 74 is a substantially continuous layer of absorbent particular polymer material across the absorbent particulate polymer material area 114 of the absorbent core 14 (i.e. first and second substrates 64 and 72 do not form a plurality of pockets, each containing a cluster 90 of absorbent particulate polymer material 66 therebetween). In a certain embodiment, respective grid patterns 92 of the first and second absorbent layer 60 and 62 may be substantially the same.

In a certain embodiment as illustrated in FIG. 8, the amount of absorbent particulate polymer material 66 and 74 may vary along the length 116 of the grid pattern 92. In a certain embodiment, the grid pattern may be divided into absorbent zones 120, 122, 124, and 126, in which the amount of absorbent particulate polymer material 66 and 74 varies from zone to zone. As used herein, “absorbent zone” refers to a region of the absorbent particulate polymer material area having boundaries that are perpendicular to the longitudinal axis shown in FIG. 8. The amount of absorbent particulate polymer material 66 and 74 may, in a certain embodiment, gradually transition from one of the plurality of absorbent zones 120, 122, 124, and 126 to another. This gradual transition in amount of absorbent particulate polymer material 66 and 74 may reduce the possibility of cracks forming in the absorbent core 14.

The amount of absorbent particulate polymer material 66 and 74 present in the absorbent core 14 may vary, but in certain embodiments, is present in the absorbent core in an amount greater than about 80% by weight of the absorbent core, or greater than about 85% by weight of the absorbent core, or greater than about 90% by weight of the absorbent core, or greater than about 95% by weight of the core. In a particular embodiment, the absorbent core 14 consists essentially of the first and second substrates 64 and 72, the absorbent particulate polymer material 66 and 74, and the SI 68 and 76. In some embodiments the absorbent core may have three or more absorbent layers. In an embodiment, the absorbent core 14 may be substantially cellulose free.

According to certain embodiments, the weight of absorbent particulate polymer material 66 and 74 in at least one freely selected first square measuring 1 cm×1 cm may be at least about 10%, or 20%, or 30%, 40% or 50% higher than the weight of absorbent particulate polymer material 66 and 74 in at least one freely selected second square measuring 1 cm×1 cm. In a certain embodiment, the first and the second square are centered about the longitudinal axis.

The absorbent particulate polymer material area, according to an exemplary embodiment, may have a relatively narrow width in the crotch area of the absorbent article for increased wearing comfort. Hence, the absorbent particulate polymer material area, according to an embodiment, may have a width as measured along a transverse line which is positioned at equal distance to the front edge and the rear edge of the absorbent article, which is less than about 100 mm, 90 mm, 80 mm, 70 mm, 60 mm or even less than about 50 mm.

It has been found that, for most absorbent articles such as diapers, the liquid discharge occurs predominately in the front half of the diaper. The front half of the absorbent core 14 should therefore comprise most of the absorbent capacity of the core. Thus, according to certain embodiments, the front half of said absorbent core 14 may comprise more than about 60% of the superabsorbent material, or more than about 65%, 70%, 75%, 80%, 85%, or 90% of the superabsorbent material.

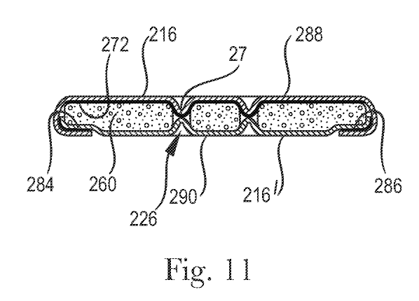

The absorbent core of the invention may comprise a core wrap enclosing the absorbent material. In some embodiments, the core wrap may be both the first and second substrates. The core wrap may be formed by two substrates, typically nonwoven material which may be at least partially sealed along the sides of the absorbent core. The first nonwoven may substantially form the top side of the core wrap and the second nonwoven substantially the bottom side of the core wrap. The core wrap may be at least partially sealed along its front side, back side and/or two longitudinal sides to improve the containment of the absorbent material during use. A C-wrap seal may be for example provided on the longitudinal sides of the core if improved containment is desired. Exemplary C-wrap description may be found in U.S. application Ser. No. 14/560,211 (Attorney docket no. CM4026). Typical core wraps comprise two substrates (216 and 216′ in FIG. 11) which are attached to another, but the core wrap may also be made of a single substrate folded around the absorbent material, or may comprises several substrates. When two substrates are used, these may be typically attached to another along at least part of the periphery of the absorbent core to form a seal. Typically neither first nor second substrates need to be shaped, so that they can be rectangularly cut for ease of production but other shapes are not excluded.

The substrates are advantageously attached to another to form a seal along all the edges of the core. Typical seals are the so-called C-wrap and sandwich wrap. In a C-wrap, such as shown in FIG. 11, one of the substrate, e.g. the first substrate 216, has flaps extending over the opposed edges of the core which are then folded over the other substrate. These flaps are bonded to the external surface of the other substrate, typically by adhesive. This so called C-wrap construction can provide benefits such as improved resistance to bursting in a wet loaded state compared to a sandwich seal.

The front side and back side of the core wrap may then also be sealed for example by adhering the first substrate and second substrate to another to provide complete enclosing of the absorbent material across the whole of the periphery of the core. For the front side and back side of the core, the first and second substrate may extend and be joined together in a substantially planar direction, forming a so-called sandwich construction. In the so-called sandwich seal construction, the first and second substrates both have material extension outwardly of the absorbent material deposition area which are then sealed flat along the whole or parts of the periphery of the core typically by gluing and/or heat/pressure bonding.

The terms “seal” and “enclosing” are to be understood in a broad sense. The seal does not need to be continuous along the whole periphery of the core wrap but may be discontinuous along part or the whole of it, such as formed by a series of seal points spaced on a line. Typically a seal may be formed by gluing and/or thermal bonding. The core wrap may also be formed by a single substrate which may enclose the absorbent material as in a parcel wrap and be for example sealed along the front side and back side of the core and one longitudinally extending seal.

The core wrap may be formed by any materials suitable for enclosing the absorbent material. Typical substrate materials used in the production of conventional cores may be used, in particular nonwovens but also paper, tissues, films, wovens, or laminate of any of these. The core wrap may in particular be formed by a nonwoven web, such as a carded nonwoven, a spunbond nonwoven (“S”) or a meltblown nonwoven (“M”), and laminates of any of these. For example spunmelt polypropylene nonwovens are suitable, in particular those having a laminate web SMS, or SMMS, or SSMMS, structure, and having a basis weight range of about 5 gsm to 15 gsm. Suitable materials are for example disclosed in U.S. Pat. No. 7,744,576, US2011/0268932A1, US2011/0319848A1, or US2011/0250413A1. Nonwoven materials provided from synthetic fibers may be used, such as polyethylene, Polyethylene terephthalate, and in particular polypropylene.

In certain embodiments, the absorbent core 14 may further comprise any absorbent material that is generally compressible, conformable, non-irritating to the wearer's skin, and capable of absorbing and retaining liquids such as urine and other certain body exudates. In such embodiments, the absorbent core 14 may comprise a wide variety of liquid-absorbent materials commonly used in disposable diapers and other absorbent articles such as comminuted wood pulp, which is generally referred to as airfelt, creped cellulose wadding, melt blown polymers, including co-form, chemically stiffened, modified or cross-linked cellulosic fibers, tissue, including tissue wraps and tissue laminates, absorbent foams, absorbent sponges, or any other known absorbent material or combinations of materials. Co-form nonwoven webs or co-form materials are known in the art and have been used in a wide variety of applications, including wipes. The term “co-form material” means a composite material containing a mixture or stabilized matrix of thermoplastic filaments and at least one additional material, often called the “second material” or “secondary material”. Examples of the second material include, for example, absorbent fibrous organic materials such as woody and non-wood pulp from, for example, cotton, rayon, recycled paper, pulp fluff; superabsorbent materials such as superabsorbent particles and fibers; inorganic absorbent materials and treated polymeric staple fibers, and other materials such as non-absorbent staple fibers and non-absorbent particles and the like. Exemplary co-form materials are disclosed in commonly assigned U.S. Pat. No. 5,350,624 to Georger et al.; U.S. Pat. No. 4,100,324 to Anderson et al.; U.S. Pat. No. 4,469,734 to Minto; and U.S. Pat. No. 4,818,464 to Lau et al.

The absorbent core 14 may further comprise minor amounts (typically less than about 10%) of materials, such as adhesives, waxes, oils and the like.

Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,207 (Dyer et al.); U.S. Pat. No. 5,397,316 (LaVon et al.); and U.S. Pat. No. 5,625,222 (DesMarais et al.).

As described, the absorbent core may comprise a first and second substrate layer that may partially enclose an absorbent layer comprising superabsorbent polymer. In some embodiments, one or both substrates may not be considered to be part of the absorbent core. In some embodiments, either or both of the substrates and/or the absorbent core may be “shaped,” meaning non-rectangular. One or both substrates and/or the absorbent core may have an I-beam shape, a “T” shape, an hourglass shape, a dumbbell shape, a mushroom shape, or any suitable shape. The absorbent core may have a central region, a front end region and a back end region, wherein the core has an average width in the central region and a relatively wider average width in at least one of the end regions. The absorbent core may be both formed and shaped on either substrate. The term “shaped” means that at least one end region of the absorbent core 14 has an average width (in the lateral direction) which is greater than the average width in the central region. The average width in the at least one end region may be at least about 5% greater, suitably at least about 10% greater, particularly at least about 25% greater, or at least about 50% greater than the average width in the central region. In one embodiment of absorbent core, both end regions have an average width which is greater, or at least about 5% greater, or at least about 10% greater, or at least about 25% greater, or at least about 50% greater than the average width of central region. For further disclosure regarding shaped cores, see U.S. Pat. No. 7,938,813.

The superabsorbent immobilizer, SI, 68 and 76 may serve to cover and at least partially immobilize the absorbent particulate polymer material 66 and 74. In one embodiment of the present invention, the SI 68 and 76 can be disposed essentially uniformly within the absorbent particulate polymer material 66 and 74, between the particles of the superabsorbent material. However, in a certain embodiment, the SI 68 and 76 may be provided as a fibrous layer which is at least partially in contact with the absorbent particulate polymer material 66 and 74 and partially in contact with the substrate layers 64 and 72 of the first and second absorbent layers 60 and 62. FIGS. 3, 4, and 7 show such a structure, and in that structure, the absorbent particulate polymer material 66 and 74 is provided as a discontinuous layer, and a layer of a fibrous thermoplastic composition or fiberized structure 68 and 76 is laid down onto the layer of absorbent particulate polymer material 66 and 74, such that the fiberized structure 68 and 76 is in direct contact with the absorbent particulate polymer material 66 and 74, but also in direct contact with the second surfaces 80 and 84 of the substrates 64 and 72, where the substrates are not covered by the absorbent particulate polymer material 66 and 74. The fiberized structures of each substrate, 68 and 76, may essentially be one fiberized structure, each contacting the other. This imparts an essentially three-dimensional structure to the fibrous structure of thermoplastic composition 68 and 76, which in itself is essentially a two-dimensional structure of relatively small thickness, as compared to the dimension in length and width directions. In other words, the thermoplastic composition 68 and 76 undulates between the absorbent particulate polymer material 66 and 74 and the second surfaces of the substrates 64 and 72, forming a fiberized structure 68 and 76.

The SI 68 and 76 may provide cavities to cover the absorbent particulate polymer material 66 and 74, and thereby immobilize the material. In accordance with certain embodiments, the SI 68 and 76 may immobilize the absorbent particulate polymer material 66 and 74 when wet, such that the absorbent core 14 achieves an absorbent particulate polymer material loss of no more than about 70%, 60%, 50%, 40%, 30%, 20%, 10% according to the Wet Immobilization Test as described in U.S. 62/158,009. Some SI may also penetrate into both the layers of absorbent particulate polymer material 66 and 74 and into the substrates 64 and 72, thus providing for further immobilization. Of course, while the SI disclosed herein provide a much improved wet immobilization (i.e., immobilization of absorbent material when the article is wet or at least partially loaded), these SI may also provide a very good immobilization of absorbent material when the absorbent core 14 is dry. In some embodiments, the SI may be a fiberized structure, a film, nanofibers, irregular blobs of material, and/or other forms.

The SI may function as a fibrous structure that entraps the absorbent particulate polymer 66 and prevents substantial movement. Materials that are most useful as a superabsorbent immobilizer include polymers with good cohesion and good elasticity or flexibility to reduce the likelihood that the superabsorbent immobilizer breaks in response to strain. In addition, the absorbent particulate polymer material will swell when wet, requiring the superabsorbent immobilizer to allow for such swelling without breaking and without imparting too many compressive forces, which would restrain the absorbent particulate polymer material from swelling. Elasticity and flexibility in the SI also promotes overall article flexibility and its preferred ability to conform to the wearer. Overall, flexible polymers having low storage modulus, i.e., G′ (as discussed later) are preferred for use in the SI. Without being bound by theory, semicrystalline polymers that have low storage modulus can also have low amounts of crystallinity. These low-crystallinity, low storage modulus polymers have an amorphous phase (defined as the remaining volume of the polymer that is not crystalline) that is elastic and rubbery at the desired temperature. A practical means to determine a polymer's level of crystallinity is by measuring its heat of fusion (melting). Polymers that have high heats of fusion are more crystalline than those that do not, so polymers with low heats of fusion are preferred for the superabsorbent immobilizer. In addition, the amorphous portion of a low-crystallinity semicrystalline polymer has greater integrity and cohesion when its molecular weight is higher and thus preserves the superabsorbent immobilizer's mechanical integrity during extension. For a superabsorbent immobilizer, polymers with relatively high molecular weight are preferred.

The absorbent core 14 may also comprise an auxiliary adhesive which is not illustrated in all the figures. The auxiliary adhesive may be deposited on the first and second substrates 64 and 72 of the respective first and second absorbent layers 60 and 62 before application of the absorbent particulate polymer material 66 and 74 for enhancing adhesion of the absorbent particulate polymer materials 66 and 74 and the SI 68 and 76 to the respective substrates 64 and 72. It may be preferable to deposit the auxiliary adhesive on a nonwoven that is the most hydrophilic for improved bonding. The auxiliary glue may also aid in immobilizing the absorbent particulate polymer material 66 and 74. The auxiliary glue may be applied to the substrates 64 and 72 by any suitable means, but according to certain embodiments, may be applied in about 0.5 to about 1 mm wide slots spaced about 0.5 to about 2 mm apart. Exemplary auxiliary adhesives include, but are not limited to, sprayable hot melt adhesives, such as H.B. Fuller Co. (St. Paul, Minn.) Product No. HL-1620-B. Other suitable auxiliary adhesives may include low-tackifier or tackifier-free adhesives such as those disclosed in U.S. Ser. No. 62/267,536 (Attorney docket No. 14128P). One thermoplastic composition may be used to provide immobilization of the absorbent particulate polymer, while an auxiliary adhesive is used in conjunction with the thermoplastic composition to adhere materials in other areas in the core. In some embodiments, a SI material may be used as an auxiliary adhesive in the core.

The SI and/or auxiliary adhesive may be applied in the absorbent particulate polymer material area at a basis weight of from about 2 grams/meter² to about 7 grams/meter² (gsm), in some embodiments, from about 5 gsm to about 15 gsm. This may be a combined basis weight from application on a first and a second substrate, for example, 4 and 3 gsm, respectively. The auxiliary adhesive may be applied in the absorbent particulate polymer material area in any amount from 0 to about 8 gsm, in some embodiments, about 5 gsm, in other embodiments about 8 gsm. The total amount of adhesive and SI may be from about 2 gsm to about 15 gsm in the absorbent particulate polymer material area. The front end seal may have from about 10 gsm to about 35 gsm of adhesive. Similarly, the back end seal may have from about 10 gsm to about 35 gsm of adhesive. In some embodiments, either or both of the front and back end seals may have from about 5 gsm to 15 gsm of adhesive. In some embodiments, the amount of adhesive in an end seal may be a combination of the the auxiliary adhesive and the end seal adhesive.

In certain embodiments, the SI 68 and 76 may be present in the form of fibers. In some embodiments, the fiberized structure will have a range of thickness from about 1 to about 90 micrometers, in some embodiments, from about 1 to about 50 micrometers, in some embodiments from about 1 to about 35 micrometers, and an average length of about 0.1 mm to about 5 mm or about 0.5 mm to about 6 mm. The average fiber thickness may be about 30 micrometers, or may be from about 20 to about 45 micrometers. Substrates 64 and 72, or any non-woven layer, may be pre-treated with an auxiliary adhesive.

The fiberized structure may consist of continuous extruded polymer strands, which create a net structure with irregular strand or filament thickness or with irregular open areas (pores or maximum strand to strand distance). Continuous polymer strands may overlap and form strand crossings or overlaps with different diameters. The applied fiberized structure may build a three-dimensional net in the absorbent core as described herein. At equivalent basis weights, a fiberized structure with thicker fibers may be more open and irregular than a fiberized structure with thinner fibers. It is believed that the thicker fibers can maintain heat in the fiber longer, which can allow the fiberized structure to wet and penetrate a nonwoven better, allowing for better stability.

If, for example, the core has channels (as discussed below) and the channels are then more secure, that is, are permanent channels, the more open structure of the fiberized structure allows the AGM or superabsorbent polymer material to adjust or move within its confined area. An exemplary SI 68 and 76, as described in more detail below, may have a storage modulus G′ measured at 25° C. of less than about 1.2×10⁹ Pa as measured by the test method described herein. The compositions of the present invention may have high G′ values, but may still be not too stiff to work as a SI or a fiberized structure in absorbent articles. A composition with a relatively high G′, such as greater than 1.2×10⁶ Pa, means a stiffer composition. It is believed that such a composition can promote thicker microfibers, and that this can aid in providing better dry absorbent polymer material stability. The net structure formed by the strands or fibers of the SI in the present invention may be less dense, thus providing more volume at the same basis weight. This is particularly true for fiberized structures comprising polyolefins.

When the absorbent article contains channels, the SI may not only help in immobilizing the absorbent material on the substrate, but it may also help in maintaining the integrity of the channels in the absorbent structure absorbent core during storage and/or during use of the disposable article. The SI may help to avoid that a significant amount of absorbent material migrates into the channels. Furthermore, when the materials are applied in the channels or on the substrate portions coinciding with the channels it may thereby help to hold the substrate of the absorbent structure to said walls, and/or to a further material, as will be described in further details below. In some embodiments, a SI may be applied as fibers, forming a fibrous network that immobilizes the absorbent material on the substrates. The thermoplastic fibers may be partially in contact with the substrate of the absorbent structure; if applied also in the channels, it (further) anchors the absorbent layer to the substrate. The thermoplastic composition material may allow for such swelling without breaking and without imparting too many compressive forces, which would restrain the absorbent polymer particles from swelling.

The cover layer 70 shown in FIG. 4 may comprise the same material as the substrates 64 and 72, or may comprise a different material. In certain embodiments, suitable materials for the cover layer 70 are the non-woven materials, typically the materials described above as useful for the substrates 64 and 72. The nonwovens may be hydrophilic and/or hydrophobic.

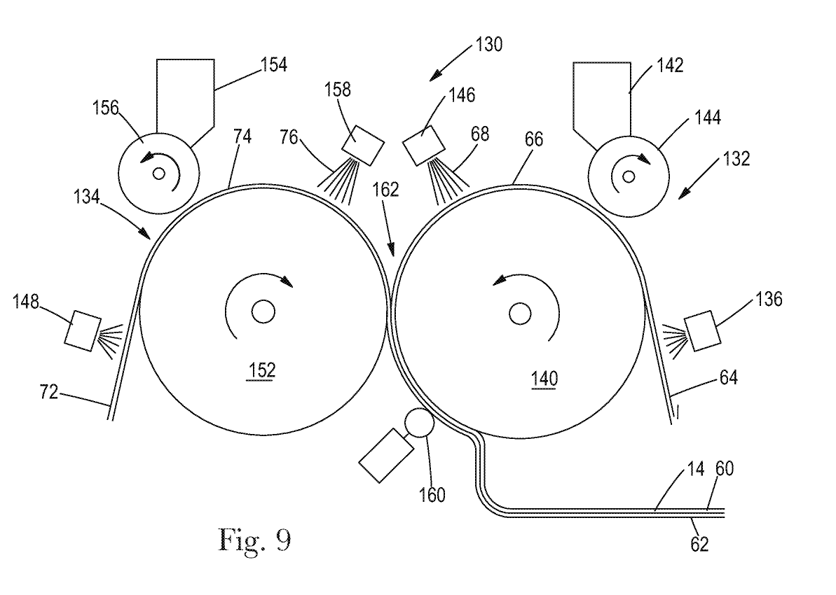

A printing system 130 for making an absorbent core 14 in accordance with an embodiment of this invention is illustrated in FIG. 9 and may generally comprise a first printing unit 132 for forming the first absorbent layer 60 of the absorbent core 14 and a second printing unit 134 for forming the second absorbent layer 62 of the absorbent core 14.

The first printing unit 132 may comprise a first auxiliary adhesive applicator 136 for applying an auxiliary adhesive to the substrate 64, which may be a nonwoven web, a first rotatable support roll 140 for receiving the substrate 64, a hopper 142 for holding absorbent particulate polymer material 66, a printing roll 144 for transferring the absorbent particulate polymer material 66 to the substrate 64, and a SI applicator 146 for applying the SI 68 to the substrate 64 and the absorbent particulate polymer 66 material thereon.

The second printing unit 134 may comprise a second auxiliary adhesive applicator 148 for applying an auxiliary adhesive to the second substrate 72, a second rotatable support roll 152 for receiving the second substrate 72, a second hopper 154 for holding the absorbent particulate polymer material 74, a second printing roll 156 for transferring the absorbent particulate polymer material 74 from the hopper 154 to the second substrate 72, and a second SI applicator 158 for applying the SI 76 to the second substrate 72 and the absorbent particulate polymer material 74 thereon.

The printing system 130 also includes a guide roller 160 for guiding the formed absorbent core from a nip 162 between the first and second rotatable support rolls 140 and 152.

The first and second auxiliary applicators 136 and 148 and the first and second SI applicators 146 and 158 may be a nozzle system which can provide a relatively thin but wide curtain of SI. In some embodiments, a contact application such as a slot gun may be used.

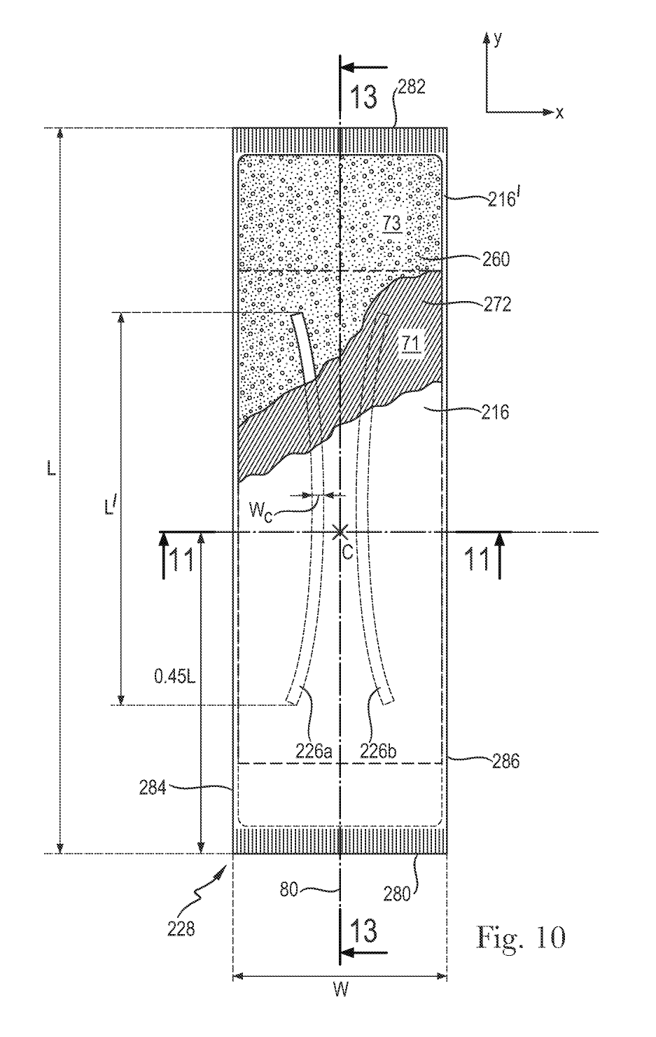

The absorbent article may further comprise a wetness indicator which is visible from the exterior of the article and which changes appearance when contacted with a body exudates, in particular urine. The wetness indicator (not shown) may be placed, when seen from the exterior of the article, between the two channel-forming areas 226a,b, of FIG. 10, and/or between any of the channel-forming areas 226a, 226b and any of the lateral edge or both. The wetness indicators of the present invention may be according to any wetness indicating system known in the art. It is known that wetness indicator can provide an appearing signal, a disappearing signal or a color change signal, and combinations thereof. The wetness indicator may advantageously provide a color change signal, which may be typically obtained by a composition having a first color when dry and a second color different form the first color when wet, both colors being discernible by an external observer considering the article in a dry and a wet state.

The wetness indicator may in particular be a color change composition comprising a suitable pH indicator or another chemical substance that changes color when contacted with urine. Such compositions are for example disclosed in WO03/070138A2 or US2012/165771 (Ruman). More generally, the wetness indicator compositions of the invention may be as disclosed in WO2010/120705 (Klofta), comprising a colorant, a matrix and a stabilizer. The color change composition may be a hot-melt adhesive, which allows for an easy application of the composition on a substrate component of the article for example by a slot coating process or printed adhesive coating as disclosed e.g. in US2011274834 (Brown). The wetness indicator composition may be applied on any layer of the absorbent article using a conventional technique, for example printing, spraying or coating, during the making of the absorbent article. The layer may advantageously be the inner surface of the backsheet or the outer surface of the bottom side of the core wrap. This allows the wetness indicator to be visible from the exterior of the article by transparency through the backsheet while keeping the wetness indicator composition within the article. The wetness indicator may in particular be easily applied on a layer such a nonwoven or film by a slot-coating process especially if the composition is can be applied as a hot-melt.

### Channels

In some embodiments, the absorbent core and/or the superabsorbent polymer material area 114 may comprise channels, or areas substantially free of superabsorbent polymer particles or any absorbent polymer material. The channels may provide improved liquid transport, and hence faster acquisition, and more efficient liquid absorbency over the whole absorbent structure, in addition to reducing the stiffness of partially or fully loaded cores.

In FIG. 10-14, an exemplary absorbent core comprises a front side 280, a back side 282 and two longitudinally extending lateral sides 284, 286 joining the front side 280 and the back side 282. The absorbent core also comprises a generally planar top side 288 and a generally planar bottom side 290 formed by the core wrap. Referring to FIG. 10, the absorbent material deposition area 73 of the core (also referred to as the superabsorbent polymer material area 114 of earlier figures) encompasses one or more area(s) 226 (e.g., 226a and 226b) which is/are substantially free of absorbent material. By “substantially free” it is meant that in each of these areas the basis weight of the absorbent material is at least less than 25%, in particular less than 20%, less than 10%, of the average basis weight of the absorbent material in the rest of the absorbent material deposition area 73 of the core. In particular there can be no absorbent material in these areas 226a and 226b. Minimal amount such as involuntary contaminations with absorbent material particles that may occur during the making process are not considered as absorbent material. The areas 226 are advantageously surrounded by the absorbent material, when considering the plane of the core, which means that the area(s) 226 does not extend to any of the edges of the deposition area 73 of the absorbent material.

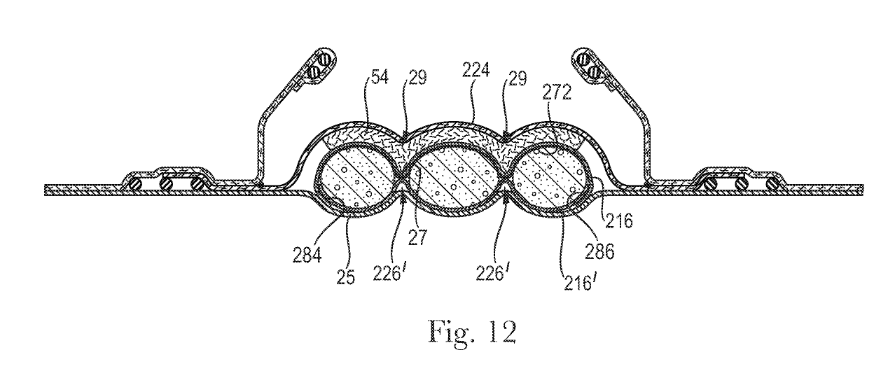

As shown for example in FIG. 11, the top side 216 of the core wrap is attached to the bottom side 216′ of the core wrap by at least one core wrap bond(s) 27 through these area(s) 226 substantially free of absorbent material. As illustrated in FIGS. 11 and 12, when the absorbent material 260 swells upon absorbing a liquid, the core wrap bond(s) 27 remain(s) at least initially attached in the substantially superabsorbent material-free area(s) 226. The absorbent material 260 swells in the rest of the core when it absorbs a liquid, so that the core wrap forms one or more channel(s) 226′ along the area(s) 226 substantially free of absorbent material comprising the core wrap bond 27. These channels 226′ are three dimensional and can serve to distribute an insulting fluid along their length to a wider area of the core. They may provide a quicker fluid acquisition speed and a better utilization of the absorbent capacity of the core. The channels 226′ can also provide a deformation of an overlying layer such as fibrous layer 54 and provide corresponding ditches 29 in the overlying layer. It is not excluded that the absorbent core may comprise other area(s) substantially free of absorbent material but without a core wrap bond, but these non-bonded areas will typically not form a channel when wet.

The inner surface of the first substrate 216 and the inner surface of the second substrate 216′ may be attached together continuously along the area(s) 226 substantially free of absorbent material, but the core wrap bond 27 may also be discontinuous (intermittent) such as formed by series of point bonds. The auxiliary glue at least partially helps forming the substrates bond 27. Typically, some pressure may be applied on the substrates in the areas 26 so that the auxiliary glue may better attach to and from the bonds between the substrates. It is also possible to additionally form the bond via other known attachment means, such as pressure bonding, ultrasonic bonding or heat bonding or combination thereof. If the auxiliary glue is applied as a series of continuous slots 272s, the width and frequency of these slots may advantageously be such that at least one slot of auxiliary glue is present at any level of the channel in the longitudinal direction. For example the slots may be 1 mm wide with a 1 mm distance between each slot, and the channel-forming area(s) have a width of about 8 mm. Such on average for 4 slots of auxiliary glue will be present in area(s) 226.

The following examples of the shape and size of the channel-forming areas 226 substantially free of absorbent material are not limiting. In general, the core wrap bond 27 may have the same outline but be slightly smaller than the areas 226 due to the tolerance required in some manufacturing process. The substantially absorbent material free area(s) 226 may be present within the crotch region of the core, in particular at least at the same longitudinal level as the crotch point C, as represented in FIG. 10 by the two longitudinally extending areas substantially free of absorbent material 226a, 226b. The absorbent core 228 may also comprise more than two substantially absorbent material free area(s), for example at least 3, or at least 4 or at least 5 or at least 6. The absorbent core may comprise one or more pairs of areas 226a, 226b substantially free of absorbent material symmetrically arranged relative to the longitudinal axis 80. Shorter area(s) substantially free of absorbent material may also be present, for example in the back region or the front region of the core, as seen for example in the Figures of WO2012/170778.

The channel-forming area(s) 226 may extend substantially longitudinally, which means typically that each area extends at least as much in the longitudinal direction (y) than in the transversal direction (x), and typically at least twice as much in the longitudinal direction than in the transverse direction (as measured after projection on the respective axis). The area(s) 226 substantially free of absorbent material may have a length L′ projected on the longitudinal axis 80 of the core that is at least 10% of the length L of the absorbent core, in particular from 20% to 80%. It may be advantageous that at least some or all of the channel-forming area(s) 226 are not completely or substantially completely transversely oriented. The area(s) substantially free of absorbent material may have a width Wc along at least part of its length which is at least 2 mm, or at least 3 mm or at least 4 mm, up to for example 20 mm, or 16 mm or 12 mm. The width Wc of the area(s) substantially free of absorbent material may be constant through substantially its whole length or may vary along its length.

The area(s) 226 substantially free of absorbent material may be completely oriented longitudinally and parallel to the longitudinal axis but also may be curved. In particular some or all these area(s), in particular these area(s) present in the crotch region, may be concave towards the longitudinal axis 80, as for example represented in FIG. 10 for the pair of channels 226a,b. The radius of curvature may typically be at least equal (and preferably at least 1.5 or at least 2.0 times this average transverse dimension) to the average transverse dimension of the absorbent material deposition area 73; and also straight but under an angle of (e.g. from 5°) up to 30°, or for example up to 20°, or up to 10° with a line parallel to the longitudinal axis. The radius of curvature may be constant for a substantially absorbent material free area(s), or may vary along its length. This may also includes area(s) substantially free of absorbent material with an angle therein, provided said angle between two parts of a channel is at least 120°, preferably at least 150°; and in any of these cases, provided the longitudinal extension of the area is more than the transverse extension. These area(s) may also be branched, for example a central substantially material free area superposed with the longitudinal axis in the crotch region which branches towards the back and/or towards the front of the article.

In some embodiments, there is no area(s) substantially free of absorbent material that coincides with the longitudinal axis 80 of the core. When present as one or more symmetrical pair(s) relative to the longitudinal axis, the area(s) substantially free of absorbent material may be spaced apart from one another over their whole longitudinal dimension. The smallest spacing distance may be for example at least 5 mm, or at least 10 mm, or at least 16 mm.

Furthermore, in order to reduce the risk of fluid leakages, the area(s) substantially free of absorbent material may advantageously not extend up to any of the edges of the absorbent material deposition area 73, and are therefore surrounded by and fully encompassed within the absorbent material deposition area 73 of the core. Typically, the smallest distance between an area(s) substantially free of absorbent material and the closest edge of the absorbent material deposition area is at least 5 mm.

The channels 226′ in the absorbent core start forming when the absorbent material absorbs a liquid such as urine and starts swelling. As the core absorbs more liquid, the depressions within the absorbent core formed by core wrap bond 27 between the two substrates will become deeper and more apparent to the eye and the touch. It is possible to create a sufficiently strong core wrap bond combined with a relatively low amount of superabsorbent polymer material and/or a relatively extensible substrate material so that the channels remain permanent until complete saturation of the absorbent material. On the other hand, the core wrap bonds may in some cases also restrict the swelling of the absorbent material when the core is substantially loaded. The core wrap bond 27 may also be designed to gradually open in a controlled manner when exposed to a large amount of fluid. The bonds may thus remain substantially intact at least during a first phase as the absorbent material absorbs a moderate quantity of fluid, as shown on FIG. 11. In a second phase the core wrap bonds 27 in the channels can start opening to provide more space for the absorbent material to swell while keeping most of the benefits of the channels such as increased flexibility of the core in transversal direction and fluid management. In a third phase, corresponding to a very high saturation of the absorbent core, a more substantial part of the channel bonds can open to provide even more space for the swelling absorbent material to expand. The strength of core wrap bond 27 within the channels can be controlled for example by varying the amount and nature of the bond used for the attaching the two sides of the core wrap, the pressure used to make the core wrap bond and/or the distribution of the absorbent material, as more absorbent material will usually causes more swelling and will put more pressure on the bond. The extensibility of the material of the core wrap may also play a role.

As shown in FIGS. 10-14, an auxiliary glue 272 is applied directly over the substrate 216 on an auxiliary glue application area 71. The auxiliary glue at least partially forms the bonds 27 between the inner surface of the first substrate 216 and the inner surface of the second substrate 216′ through the area(s) 226a,b substantially free of absorbent material. The auxiliary glue 272 may also be useful to improve the adhesion between the first substrate 216 and both the absorbent material (in the absorbent material land areas 75) and the SI 274 (in the absorbent material-free junction areas 276).

The “auxiliary glue application area” as used herein means the smallest area 71 in the plane of the substrate 216 whose periphery encompasses the auxiliary glue 272 and any areas free of auxiliary glue between the auxiliary glue. The auxiliary glue application area 71 is smaller than the absorbent material deposition area 73 (superabsorbent polymer material area). The auxiliary glue may thus be advantageously be applied in the area of the first substrate 216 where it is most needed, foremost where the channel-forming region(s) 226a,b are present and a bond 27 between the two substrates is desired, and typically at or close to the crotch region of the absorbent core as well where the amount of absorbent material may be typically higher than in the back region of the core. Reducing the auxiliary glue application area 71 relative to the absorbent material deposition area 73 has the advantage that typically less auxiliary glue material is used compared to a full application area. Reducing the amount and area of the auxiliary glue may also provide improved fluid acquisition properties as hotmelt glue are typically hydrophobic as well as reduced undesired glue smell in the finished product.

In general, the auxiliary glue application area may be at least 20% smaller than the absorbent material deposition area 73, in particular from 20% to 80% smaller than the absorbent material deposition area 73. The areas are compared by measuring their surface in the plane of the absorbent core and including the channel-forming area 226′ in the absorbent material deposition area 73.

The auxiliary glue application area may be shorter in the longitudinal direction (y) and/or in the transversal direction (x) than the absorbent material deposition area 73. The auxiliary glue application area 71 may be for example generally rectangular and have about the same width as the absorbent material deposition area 73 while being shorter in the longitudinal direction (y). FIG. 10 shows such an example where the auxiliary glue application area 71 and absorbent material deposition area 73 are both rectangular, have the about the same width and wherein the application area 71 is longitudinally shorter than the deposition area 73 and does not extend to any of the front or back ends of the absorbent material deposition area. An alternative configuration may be where the auxiliary glue application area 71 is shorter in both longitudinal and transversal directions than the absorbent material deposition area 73. Of course many different configurations for the both areas are possible, as the absorbent material deposition area 73 may also be shaped instead of rectangular. The auxiliary glue application area 71 may also for example extend from the front end of the absorbent material deposition area 73 and along its width and stop before the back end of the absorbent material deposition area. This may be advantageous for application having a relatively high amount of superabsorbent polymer material towards the front of the core, where the auxiliary glue may be needed there. The auxiliary glue application area may also have a shape which is not rectangular but for example having a central body with two adjoined side wings which are shorter than the central body. The wings may or may not extend to the lateral edges of the absorbent material deposition area but they may also extend to these edges if desired. These sections of different lengths may for example be easily obtained using a slot coating process and tuning the slot nozzles to apply the hot-melt adhesive on a shorter distance on the sides of the application area compared to the center of the application area.

The auxiliary glue application area 71 may have any shape adapted to the intended usage of the absorbent article and the distribution of absorbent material. In particular, the auxiliary glue application area may be rectangular, shaped with a tapering in the central region of the substrate, or with a central elongated portion and shorter side portions. It is also possible that the auxiliary glue application area comprises separated sub-areas. A sub-area is hereby defined as an adhesive application area separated from another at least about 10 mm. In that case the adhesive free area between the adhesive application sub-areas is not considered to be part of the auxiliary glue application area, for example for the determination of the surface of the auxiliary glue area 71. In such a configuration, where the auxiliary glue application area 71 consists of two sub-zones, each of these zones generally corresponding to one channel-forming area 226a, 226b and separated by a distance of about 10 mm.

In the above description, the auxiliary glue 272 was discussed with reference to the first absorbent substrate 216 which forms the upper side 288 of the absorbent core, and which is placed towards the topsheet 224 in the finished absorbent article 20. This is however not limiting, as the first substrate may alternatively form the bottom side 290 of the absorbent core which is placed towards the backsheet 25 of the article 20. It is also considered that a second auxiliary glue may be applied directly on the second substrate in addition to the first auxiliary glue applied directly on the first substrate, in particular in any of the configurations discussed above. This may be particular useful when the absorbent material within the core wrap comprises two layers as discussed above.

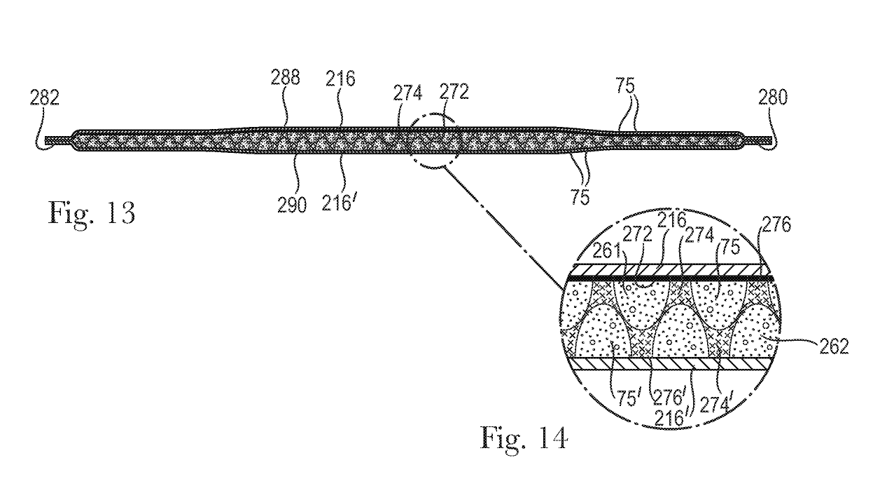

As shown in FIGS. 13 and 14, the absorbent core may also comprise a superabsorbent immobilizer (SI) 274, which may be a fibrous thermoplastic material, to further immobilize the absorbent material 261 and 262 during the making process of the core and usage of the article. This SI 274, 274′ may be in particular useful to immobilize the layer of absorbent materials 261, 262 to their respective substrate 216, 216′. These absorbent layer(s) may comprise land areas 75, 75′ separated by junction areas 276, 276′ as discussed above and the SI 274 may then be at least partially in contact with the absorbent material 261, 262 in the land areas and at least partially in contact with the substrate layer 216, 216′ in the junction areas. This imparts an essentially three-dimensional net-like structure to the SI, which in itself may be essentially a two-dimensional structure of relatively small thickness, as compared to the dimension in length and width directions. Thereby, the SI may provide cavities to cover the absorbent material in the land areas, and thereby immobilizes this absorbent material. The SI may be for example sprayed on an absorbent layer after it has been deposited on its substrate during the core making process.

### SI—Superabsorbent Immobilizer

The SI of the present invention is a composition that is applied to the superabsorbent material with the intent to immobilize the superabsorbent material in both the dry and wet state. The SI may be a fiberized structure with, for example, microfibers or nanofibers, or may be a film, or discrete blobs of material, or some other form. The SI may be, for example, a discrete form such as a layer of material, or a fibrous material that interweaves or intertwines with surrounding materials, such as the particles of superabsorbent material. A single absorbent core may have more than one SI, if the SI is in a countable form such as discrete layers. A single absorbent core may have more than one area comprising SI, and some of the SI from each area may or may not have contact or intertwine. A single absorbent core may have more than one form of SI.

The SI of the present invention may comprise little to nothing other than high molecular weight (MW) polymer material. In some embodiments, the superabsorbent immobilizer may consist essentially of one or more polymers having a peak molecular weight of at least about 10 kg/mol. In some embodiments, the superabsorbent immobilizer may comprise at least about 55%, 60%, 65%, 70%, 75%, 80%, 85%, 90%, 95%, 97%, 98%, 99%, 100% by weight of one or more polymers each having a peak molecular weight of at least about 10 kg/mol. In some embodiments, the SI may comprise at least one polymer having a peak molecular weight of at least about 10 kg/mol, as determined using the gel permeation chromatography method described herein, wherein the combined total of each of these high molecular weight polymers comprises at least 50% by weight of the superabsorbent immobilizer.

The superabsorbent immobilizer may comprise at least one component whose peak molecular weight is less than about 9.0 kg/mol. In some embodiments, the superabsorbent immobilizer may comprise at most about 50% by weight of these components with peak molecular weights less than about 9.0 kg/mol. In some embodiments, the superabsorbent immobilizer may comprise at most about or less than about 45%, 40%, 35%, 30%, 25%, 20%, 15%, 10%, 5%, 3%, 2%, or 1% by weight of components having a peak molecular weight less than about 9.0 kg/mol. These components with lower peak molecular weights may be additives, such as tackifiers, plasticizers, oils, waxes, surfactants, crystallinity enhancers, and/or other materials typically mixed with high molecular weight polymers in order to be used in hotmelt compositions for absorbent articles, as are known in the art. In some embodiments, the superabsorbent immobilizer may be substantially tackifier-free, in some embodiments, it may comprise at most about 40%, 35%, 30%, 25%, 20%, 15%, 10%, 5%, 3%, 2%, or 1% tackifier, and in some embodiments may comprise 0% tackifier. In some embodiments, the superabsorbent immobilizer may be substantially free of any tackifiers, waxes, oils, and plasticizers.

The superabsorbent immobilizer may have a storage modulus (G′) at 25° C. of less than about 1.2×10⁹ Pa. The high molecular weight polymers, i.e., those with a peak molecular weight of at least about 10 kg/mol, may be the sole material or a high percentage of the superabsorbent immobilizer, and may also have a storage modulus (G′) at 25° C. of less than about 1.2×10⁹ Pa.

The superabsorbent immobilizer may have a heat of fusion value of less than about 80 J/g. In some embodiments, the heat of fusion of the superabsorbent immobilizer may be less than about 50 J/g, less than about 40 J/g, less than about 35 J/g, from about 2 to about 80 J/g, from about 5 to about 50 J/g, or from about 7 to about 35 J/g. The high molecular weight polymers may also have heat of fusion values of less than about 80 J/g, less than about 50 J/g, less than about 40 J/g, from about 2 to about 80 J/g, from about 5 to about 50 J/g, or from about 7 to about 35 J/g.

The superabsorbent immobilizer may comprise high molecular weight polymers (i.e., those with peak molecular weights of at least about 10 kg/mol) selected from the group consisting of polymers and copolymers of propylene, ethylene, butene, and combinations thereof; styrenic block copolymers; polyolefins; olefin block copolymers, and combinations thereof. Additional suitable polymers include polyolefins (e.g., homopolymers, copolymers, or terpolymers of C2 to C8 alphaolefins, polyethylenes, polypropylenes, polypropylene-co-ethylene, polyethylene-co-propylene, polypropylene-co-butene, polyethylene-co-octene, branched, linear, HDPE, LDPE, LLDPE, metallocene catalyzed, Ziegler-Natta catalyzed, living polymerizations, etc.) and blends, mixtures, copolymers, thereof; polyethylene/unsaturated carboxylic acid, salt and ester interpolymers (e.g., polyethylene vinyl acetate (EVA), polyethylene ethyl-acrylate (EEA), polyethylene/methyl acrylate, polyethylene isobutyl acrylate, polyethylene methyl methacrylate) and blends, mixtures thereof; polyesters (e.g., aliphatic polyesters, poly(ethylene terephthalate), poly(ethylene isophthalate), poly(hydroxyalkanoates), polylactic acid, polyglycolic acid) and blends, mixtures and copolymers thereof; polystyrenes (e.g., poly(alpha-methystyrene), poly(2-methyl styrene), poly(4-methyl styrene), poly(4-methoxystyrene), poly(4-phenyl styrene), poly(4-pheny-1-propene), and blends, mixtures and copolymers thereof; polyacrylates (e.g., poly(acrylic acid), poly(methy acrylate), poly(ethy acrylate), poly(propyl acrylate), poly(isopropyl acrylate), poly(butyl acrylate), poly(isobutyl acrylate), and blends, mixtures and copolymers thereof; styrenic block copolymers (e.g., ABA triblock structures, A-B diblock structures and (A-B)n radial structures wherein the A blocks are non-elastomeric polymer blocks, typically comprising polystyrene, and the B blocks are unsaturated conjugated diene or partly hydrogenated versions of such. The B block is typically polyisoprene, polybutadiene, poly-ethylene/butane (hydrogenated polybutadiene), poly-ethylene/propylene (hydrogenated polyisoprene) and blends, mixtures thereof. Preferred superabsorbent immobilizers include homopolymers, copolymers, or terpolymers of C2 to C8 alphaolefins, and blends or mixtures thereof. Even more preferred superabsorbent immobilizers include metallocene catalyzed polypropylene copolymerized with ethylene. Examples of polymers that may be all or part of a superabsorbent immobilizer include Vistamaxx 8380 and Vistamaxx 8780, commercially available from Exxon. In some cases, the entire superabsorbent immobilizer may consist essentially of one material, such as the Vistamaxx 8380 or Vistamaxx 8780.

The superabsorbent immobilizers may be soluble in organic solvents at room temperature or elevated temperature. The peak molecular weight of each polymer in the superabsorbent immobilizer may be less than about 3,000 kg/mol. Each of the high molecular weight polymers may be used alone as a superabsorbent immobilizer, or may be combined with other polymers. Some superabsorbent immobilizers may be substantially 100% high molecular weight polymer, either a single high molecular weight polymer or a combination of two or more high molecular weight polymers. Some superabsorbent immobilizers may be one high molecular weight polymer or a combination of high molecular weight polymers combined with additives, such as some amount of tackifier, plasticizer, oil, waxes, and/or other materials as are known in the art. Any additive may be considered a low molecular weight material, meaning a molecular weight less than about 9.0 kg/mol.

In some embodiments, functionalized polymers, such as functionalized polyolefins, may be used as all or part of the superabsorbent immobilizer. Such functional polymers may be hydrophilic, and thus be advantageous. A fibrous nonwoven web or web-forming material may be chemically reacted with a polar material. The polar material can include an anhydride or anhydride derivative (e.g., a carboxylic acid derivative) and can be a monomer, polymer, or compound. The reaction product is a hydrophobic polymer material having a polar functionality (herein called a polar-modified polymer). Preferably, the nonwoven web or web-forming material is reacted with maleic anhydride or one of its derivatives, such as maleic acid or fumaric acid. Other suitable polar materials include without limitation various anhydrides and their derivatives, particularly those having an unsaturated carbon-carbon double bond: HOOCCH═CHCOOH. The polar material is reacted with the hydrophobic polymer, either using heat or a catalyst (e.g., a peroxide catalyst), or a combination of heat and catalyst. When heat is employed, the reaction may take place at a temperature near or above the melting point of the hydrophobic polymer. For instance, the hydrophobic polymer and polar material may be blended together in a mixer, with the hydrophobic polymer in the molten state, to facilitate substantially homogeneous mixing and reaction between the polar material and hydrophobic polymer. When the hydrophobic polymer includes polypropylene, for instance, the reaction may occur in a mixer at a temperature of about 160-225° C., preferably 175-200° C., with or without a peroxide catalyst, whereby the polar material is graft polymerized onto the hydrophobic polymer. Alternatively, the chemical reaction may occur at a much lower temperature in a solvent, with the grafting reaction being aided by a peroxide catalyst.

Techniques for graft polymerizing a polar material, such as maleic anhydride or a dicarboxylic acid derivative, onto a hydrophobic polymer (e.g., a polyolefin) are well known in the art. As an alternative to polymerizing the polar material with the hydrophobic polymer, a suitable polar-modified hydrophobic polymer may be purchased commercially. Commercially available polar-modified hydrophobic polymers include without limitation the following: EXXELOR® 1015, a maleated polypropylene available from Exxon Chemical Co., having a melt flow rate (230° C.) of 120 grams/10 minutes and containing 0.4% by weight grafted maleic anhydride; POLYBOND® 3150, a maleated polypropylene available from Uniroyal Chemical Co., having a melt flow rate (230° C.) of 50 grams/10 minutes and containing 0.7% by weight grafted maleic anhydride; and POLYBOND® 3200, a maleated polypropylene available from Uniroyal Chemical Co., having a melt flow rate (230° C.) of 110 grams/10 minutes and containing 1.0% by weight grafted maleic anhydride.

The maleated polyolefin (or other polar-modified polymer) may itself be hydrophobic and not wettable to water, or borderline between hydrophobic and hydrophilic. The reaction with the polar material does not render the polymer backbone hydrophilic; rather, it provides a chemical linkage for the subsequent reaction with a hydrophilic material. Generally, the polar-modified polymer should contain about 0.1-3.0% by weight of the polar monomer, preferably about 0.4-1.0% by weight, more preferably about 0.6-0.8% by weight. Preferably, the polar material is grafted onto the hydrophobic polyolefin, resulting in a stereochemistry most favorable for further reaction.

The polar-modified hydrophobic polymer may be reacted with a hydrophilic material, thereby increasing the hydrophilicity of the polymer to render it wettable to water. The hydrophilic material can be a hydrophilic monomer, polymer, compound, or blend containing one or more of these. Suitable hydrophilic materials include organic alcohols, dialcohols, tertiary alcohols, polymers containing them, and other hydrophilic materials having groups which react with the polar group (e.g., the anhydride moiety) on a polar-modified hydrophobic polymer. Other suitable hydrophilic materials include polyglycols and polyoxides, including polyolefin glycols and oxides, such as polyethylene glycol, polyethylene oxide, polypropylene glycol, polypropylene oxide, and copolymers and mixtures thereof. Polyglycols include those having monoamine and/or diamine linkages which further promote hydrophilicity. The JEFF AMINE® series of polyglycols, available from Huntsman Chemical Co., includes monoamines and diamines of varying molecular weights. For further disclosure concerning the reaction between a polar-modified hydrophobic polymer and a hydrophilic material and disclosure in general about suitable functionalized polymers, see WO 2000012801.

As used herein, the term “tackifying resin” or “tackifier” includes:

(a) aliphatic and cycloaliphatic petroleum hydrocarbon resins having Ring and Ball softening points of from 10° C. to 160° C., as determined by ASTM method E28-58T, the latter resins resulting from the polymerization of monomers consisting primarily of aliphatic and/or cycloaliphatic olefins and diolefins; also included are the hydrogenated aliphatic and cycloaliphatic petroleum hydrocarbon resins; examples of such commercially available resins based on a C5 olefin fraction of this type are Piccotac tackifying resin sold by Eastman Chemical Company and Escorez 131OLC sold by ExxonMobil Chemical Company;

(b) Aromatic petroleum hydrocarbon resins and the hydrogenated derivatives thereof;

(c) Aliphatic/aromatic petroleum derived hydrocarbon resins and the hydrogenated derivatives thereof;

(d) Aromatic modified cycloaliphatic resins and the hydrogenated derivatives thereof;

(e) Polyterpene resins having a softening point of from about 10° C. to about 140° C., the latter polyterpene resins generally resulting from the polymerization of terpene hydrocarbons, such as the mono-terpene known as pinene, in the presence of Friedel-Crafts catalysts at moderately low temperatures; also included are the hydrogenated polyterpene resins;

(f) Copolymers and terpolymers of natural terpenes, e.g. styrene/terpene, a-methyl styrene/terpene and vinyl toluene/terpene;

(g) natural and modified rosin such as, for example, gun rosin, wood rosin, tall-oil rosin, distilled rosin, hydrogenated rosin, dimerized rosin and polymerized rosin;

(h) glycerol and pentaerythritol esters of natural and modified rosin, such as, for example, the glycerol ester of pale wood rosin, the glycerol ester of hydrogenated rosin, the glycerol ester of polymerized rosin, the pentaerythritol ester of pale wood rosin, the pentaerythritol ester of hydrogenated rosin, the pentaerythritol ester of tall-oil rosin, and the phenolic modified pentaerythritol ester of rosin;

(i) phenolic-modified terpene resins such as, for example, the resin product resulting from the condensation in an acidic medium of a terpene and a phenol.

Mixtures of two or more of the above described tackifying resins may be required for some formulations. Tackifying resins which are useful for the present invention can also include polar tackifying resins. Suitable resins are aliphatic petroleum hydrocarbon resins examples of which are based on C5 olefins such as Hercotac 1148 available from Hercules Corp. Also suitable are nonpolar products which are hydrogenated dicyclopentadiene (DCPD) based or aromatically modified derivatives thereof with softening points above 70° C. Examples of such resins are Escorez 5400 and Escorez 5600 sold by ExxonMobil Chemical Company. Tackifiers may be present in superabsorbent immobilizer in amounts of 0% to 50% by weight, in some embodiments, from 5% to about 15%, in some embodiments, less than about 20% or less than about 10%.

A plasticizer can be present in the compositions of the present invention in amounts of 0% to about 10% by weight. A suitable plasticizer may be selected from the group which includes the usual plasticizing oils, such as mineral oil, but also olefin oligomers and low molecular weight polymers, as well as vegetable and animal oils and derivatives of such oils. The petroleum derived oils which may be employed are relatively high boiling materials containing only a minor proportion aromatic hydrocarbons. In this regard, the aromatic hydrocarbons should preferably be less than 30% and more particularly less than 15% of the oil, as measured by the fraction of aromatic carbon atoms. More preferably, the oil may be essentially non-aromatic. The oligomers may be polypropylenes, polybutenes, hydrogenated polyisoprenes, hydrogenated polybutadiens, or the like having average molecular weight between about 350 and about 9,000. Suitable vegetable and animal oils include glycerol esters of the usual fatty acids and polymerization products thereof. Other useful plasticizers can be found in the families of conventional dibenzoate, phosphate, phthalate esters, as well as esters of monoorpolyglycols. Examples of such plasticizers includes, but are not limited to dipropylene glycol dibenzoate, pentaerythritoltetrabenzoate, 2-ethylhexyl diphenyl phosphate, polyethylene glycol 400-di-2-ethylhexoate; butyl benzyl phthalate, dibutyl phthalate and dioctylphthalate.

A wax may be present in the superabsorbent immobilizer in amounts from 0% to about 35%, from about 5% to about 30%, or from about 10% to about 25%. The wax may be any of those conventionally used in hot melt compositions. Exemplary petroleum derived synthetic waxes are paraffin and microcrystalline waxes having melting points within a range of from about 55° C. to about 110° C., as well as low molecular weight polyethylene and Fischer-Tropsch waxes.

A surfactant used may have an HLB of less than 15, the said surfactant consisting of a fatty acid ester incorporated into the composition in an amount such that the resultant composition has a contact angle of 75° or less, and preferably less than about 40°. Contact angle measurements of liquid droplets on substrate surfaces are used to characterize surface wettability. The contact angle is defined as the angle between the substrate support surface and the tangent line at the point of contact of the liquid droplet with the substrate. The value of the contact angle of the liquid droplet will depend upon the surface energy of the substrate and the surface tension of the liquid. If complete wetting takes place between the liquid and the substrate surface, the droplet will spread out over the substrate and the contact angle will approach zero, whereas if wetting is only partial, the resulting contact angle will lie in the range of 0 to 180 degrees.

A low contact angle is desirable so that water, urine or other water-based discharges “wet out” rather than “bead up”. The lower the contact angle, the more hydrophilic is the material. The water contact angle may be measured by ASTM D5946-96.

Additional examples of suitable surfactants include, but are not limited to, the following: (1) Fatty acid esters such as glycerol esters, PEG esters, and sorbitan esters, including ethylene glycol distearate, ethylene glycol monostrearate, glycerol mono and/or dioleate, PEG dioleate, PEG monolaurate, sorbitan monolaurate, sorbitan trioleate, etc. These surfactants are available from ICI, Rhone-Poulenc, and other sources; (2) Nonionic ethoxylates, such as alkylphenol ethoxylates, alcohol ethoxylates, alkylamine ethoxylates, etc., including octylphenol ethoxylate, nonylphenol ethoxylate, alkylamine ethoxylates, etc. These surfactants are available from Rhone-Poulenc, Union Carbide, and other sources; (3) Nonionic surfactants such as 2,4,7,9-tetramethyl-5-decyn-4,7-diol available from Air Products; (4) Ethylene oxide/Propylene oxide copolymers, which are available from Union Carbide, BASF, etc.; (5) Atmer 688, a nonionic surfactant blend, and Alkamuls GMS/C a glycerol monostearate, both manufactured by ICI Americas Inc. It should be noted that these and other surfactants can be blended if necessary to produce the best blend of hydrophilic performance properties. Other suitable surfactants may be found in U.S. Pat. No. 6,380,292.

Crystallinity enhancers that may be added to the superabsorbent immobilizer include, but are not limited to, microcrystalline waxes and crystalline olefin homopolymers. In particular, a linear polyethylene homopolymer may be used. Without being bound by theory it is believed that such materials crystallize more readily as temperature decreases due to minimal branching. The crystallites thus formed then serve as a template for crystallization of the polymeric material. In addition, crystallinity enhancers can be very small solid particles that act as a foreign phase providing a new surface on which crystal growth can occur. This foreign phase takes the form of a nucleating agent which has been designed to have a good epitaxial match with the growing polymer crystal. A uniform dispersion of these particles increases the crystallization nucleation and growth. Nucleation agents may be helpful for speeding up the transformation of the superabsorbent immobilizer from the molten state when it is applied to the superabsorbent. Examples of nucleation agents that work as crystallinity enhancers include inorganic and ceramic powders such as zirconia, calcium carbonate, magnesium silicate, silica gels, clays such as bentonite, metal oxides, and their organically modified versions thereof. Organic materials and salts can also work as nucleation agents, examples are aromatic carboxylic acid salts, sodium benzoate, and certain pigment colorants. Commercial examples of nucleation agents include the Hyperform® products from Milliken. Crystallinity enhancers may comprise less than about 5% of the superabsorbent immobilizer, in some cases, about 1% to 5%, or about 0.1% to about 1%, about 0.5% to about 2%, or from about 2% to about 4%.

### Examples

Each of the materials below, Examples 1-7 in Table 1, may be used as an exemplary superabsorbent immobilizer. That is, each exemplary superabsorbent immobilizer is 100% of the materials listed below with no additives. Listed for each material is its G′, peak molecular weight, and heat of fusion.

TABLE 1

| | | G′ | Peak Molecular | Heat of |
|---|---|---|---|---|
| | | at ~25° C. | Weight | Fusion |
| | Material | (Pa) | (g/mol) | (J/g) |
| Example 1 | Lyondel Basell | 2.7E+07 | 89,000 | 102 |
| | PP MF650X | | | |
| Example 2 | Lyondel Basell | 5.2E+07 | 121,000 | 101 |
| | PP MF650W | | | |
| Example 3 | Lyondel Basell | 2.3E+08 | 251,000 | 96 |
| | PP PH835 | | | |
| Example 4 | Exxon | 5.8E+05 | 182,000 | 1 |
| | Vistamaxx | | | |
| | 6502 | | | |
| Example 5 | Exxon | 2.2E+07 | 43,000 | 44 |
| | Vistamaxx | | | |
| | 8880 | | | |
| Example 6 | Dow Affinity | 3.7E+06 | 50,000 | 70 |
| | GA1950 | | | |
| Example 7 | Dexco Vector | 5.0E+07 | 86,000 | 4.3 |
| | 4411A | | | |

In some cases, a superabsorbent immobilizer may comprise a combination of two or more high molecular weight polymers, with no low molecular weight material (below 9.0 kg/mol) and/or additives, such as in Example 8 in Table 2 below. And in some cases, a superabsorbent immobilizer may be one or more high molecular weight polymers combined with polymers, additives, and/or other components whose molecular weights are below 9.0 kg/mol, such as Examples 9-12 in Table 2 below, where the Escorez grades are hydrogenated (5300) and less hydrogenated tackifiers (5600), the Indopol is a polybutene, and the Calsol is an oil.

TABLE 2

| | Exam- | Exam- | Exam- | Exam- | Exam- |
|---|---|---|---|---|---|
| | ple 8 | ple 9 | ple 10 | ple 11 | ple 12 |
| Exxon | | 60% | | | |
| Vistamaxx | | | | | |
| 6502 | | | | | |
| Exxon | 50% | | | | |
| Vistamaxx | | | | | |
| 8880 | | | | | |
| Dexco Vector | | | 70% | | |
| 4411A | | | | | |
| Exxon | | | | 50% | |
| Vistamaxx | | | | | |
| 6202 | | | | | |
| Idemitsu | | | | | 55% |
| L-MODU | | | | | |
| S400 | | | | | |
| Clariant | 50% | | | | |
| Licocene | | | | | |
| PP1602 | | | | | |
| Exxon Escorez | | | 25% | | |
| 5600 | | | | | |
| Exxon Escorez | | 35% | | 40% | 40% |
| 5300 | | | | | |
| Ineos Indopol | | 5% | | | 5% |
| H-100 | | | | | |
| Calumet Calsol | | | | 10% | |
| 550 | | | | | |

### Test Methods

### Molecular Weight

Molecular weight, as used herein, refers to peak molecular weight, as determined using a gel permeation chromatography (GPC) method. GPC is a well-known method wherein polymers are separated according to molecular size, the largest molecule eluting first. The chromatograph is calibrated using commercially available polystyrene molecular weight standards. The peak molecular weights referred to herein can be determined with gel permeation chromatography (GPC) using polystyrene calibration standards, such as is done according to ASTM D5296. The molecular weight of any polymer or unknown polymer measured using GPC so calibrated is the styrene equivalent molecular weight, which herein is defined as the “peak molecular weight.” Suitable solvents and temperatures are employed with GPC in order to achieve adequate molecular weight separation and resolution.

### Heat of Fusion

Heat of fusion, as used herein, is determined using ASTM D3418-08.

### G′: Dynamic Mechanical Analysis (DMA) to Determine G′ for Thermoplastic Compositions

Temperature Sweep—Principle

A dynamic mechanical analysis (DMA) is done. An oscillatory shear stress is continuously applied to the composition resulting in an oscillatory strain at constant amplitude, which is small enough to ensure fully recoverable deformation, whereas the temperature is decreased in discrete steps. The relationship between the sinusoidal stress applied and the resulting strain response as well as the shift between both measures on the time axis are measured. The results are quantified by Storage Modulus [G′], Loss Modulus [G″] and Loss Factor [tan δ] of the composition in dependence of temperature.

### Instrument:

### TA Instruments AR G2

### Procedure:

1. Use a rheometer with 8 mm plate/plate geometry consisting of an upper steel plate (diameter: 8 mm) and a lower peltier or heating plate enabling temperature control. The rheometer needs to be capable of applying temperatures from 0° C. to 220° C.
2. Calibrate Rheometer according to instrument manual.
3. Cut off and weigh a piece of composition of 0.37 g+/−0.01 g and place it onto the centre of the Peltier or heating plate of the rheometer and set the temperature to 160° C.
4. After the composition is molten, slowly lower the upper plate to the geometry gap of 1000 micrometer. The velocity of the rheometer head must not exceed 1000 micrometer per second in order to achieve good contact between the composition and the upper plate without damaging the composition sample.
5. Cover the geometry with the geometry cover for 2 minutes so that the upper plate can heat up and the composition gets completely molten.
6. Remove the cover and rotate the upper plate manually to distribute the composition evenly between the upper plate and the Peltier or heating plate and to ensure full contact of the composition to the upper plate.
7. Afterwards cover the geometry with the geometry cover for another 2 minutes.
8. Remove the geometry cover and check whether the composition is distributed evenly.
9. Set Axial force control to 0.2 N with a sensitivity +/−0.1 N
   Start Temperature Sweep from 160° to 0° C. with a temperature ramp of 3° C. per minute. Strain

### Amplitude: 0.05%

Angular frequency: 6.28319 rad/s

### Calculation/Reporting

From the temperature sweep report the following parameters:
Glass transition temperature in ° C.
   (The glass transition temperature is defined at the peak maximum of the tan δ value
Cross-over temperature in ° C.
   (The cross-over-temperature is found at the end of the rubber-plateau towards higher temperatures indicating the beginning of the terminal zone. At the cross-over-temperature storage- and loss modulus equal and tan δ value is 1)
Storage modulus at 25° C., 35° C., 60° C. and 90° C. in Pascal.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. An absorbent article comprising an absorbent core;
said absorbent core comprising superabsorbent polymer material and a superabsorbent immobilizer;
said superabsorbent immobilizer comprising at least about 50% by weight of one or more polymers each having a peak molecular weight of at least about 10 kg/mol, as determined using the gel permeation chromatography method described herein.

2. The absorbent article of claim 1, wherein the superabsorbent immobilizer further comprises at least one component that has a peak molecular weight less than about 9.0 kg/mol, as determined using the gel permeation chromatography method described herein.

3. The absorbent article of claim 2, wherein all the components with a peak molecular weight less than about 9.0 kg/mol comprise less than about 10% by weight of the superabsorbent immobilizer.

4. The absorbent article of claim 1, wherein the superabsorbent immobilizer is substantially tackifier-free.

5. The absorbent article of claim 1, wherein the superabsorbent immobilizer has a storage modulus (G′) at 21° C. of less than about 1.2×10⁹ Pa.

6. The absorbent article of claim 1, wherein the superabsorbent immobilizer has a heat of fusion value of less than about 80 J/g.

7. The absorbent article of claim 1, wherein the superabsorbent immobilizer is substantially free of plasticizers, oils, and waxes.

8. The absorbent article of claim 1, wherein the superabsorbent immobilizer comprises polymers selected from the group consisting of polymers and copolymers of propylene, ethylene, butene, and combinations thereof; styrenic block copolymers; polyolefins; olefin block copolymers, and combinations thereof.

9. The absorbent article of claim 1, wherein the one or more polymers having a peak molecular weight of at least about 10 kg/mol comprise at least about 90% by weight of the superabsorbent immobilizer.

10. The absorbent article of claim 1, wherein the one or more polymers having a peak molecular weight of at least about 10 kg/mol comprise at least about 98% by weight of the superabsorbent immobilizer.

11. The absorbent article of claim 1, wherein the superabsorbent immobilizer further comprises at least one component that has a peak molecular weight less than about 9.0 kg/mol, and such components comprise less than about 1% of the superabsorbent immobilizer.

12. The absorbent article of claim 1, wherein the superabsorbent immobilizer is a fiberized structure.

13. The absorbent article of claim 12, wherein the superabsorbent immobilizer intertwines with the superabsorbent polymer material.

14. The absorbent article of claim 1, wherein the absorbent article further comprises a topsheet and a backsheet, with the absorbent core disposed therebetween.

15. The absorbent article of claim 1, wherein the absorbent core comprises a first absorbent layer comprising a first substrate, at least a portion of the superabsorbent polymer material deposited on said first substrate, and at least a portion of the superabsorbent immobilizer covering the superabsorbent polymer material, and wherein the first substrate is a nonwoven core cover.

16. The absorbent article of claim 15, wherein the absorbent core further comprises a second absorbent layer comprising a second substrate, at least a portion of the superabsorbent polymer material deposited on said second substrate, and at least a portion of the superabsorbent immobilizer covering the superabsorbent polymer material, wherein said first and second absorbent layers are combined together such that at least a portion of the superabsorbent immobilizer of the first absorbent layer contacts at least a portion of the superabsorbent immobilizer of the second absorbent layer.

17. The absorbent article of claim 16, wherein the second substrate is at least partially bonded to the first substrate and wherein the second substrate is a nonwoven core cover.

18. An absorbent article comprising an absorbent core;
wherein the absorbent core comprises first and second absorbent layers, the first absorbent layer comprising a first substrate and the second absorbent layer comprising a second substrate;
wherein the first and second absorbent layers further comprise superabsorbent polymer material deposited on said first and second substrates and a superabsorbent immobilizer covering the superabsorbent polymer material on the respective first and second substrates;
wherein said first and second absorbent layers are combined together such that at least a portion of the superabsorbent immobilizer of the first absorbent layer contacts at least a portion of the superabsorbent immobilizer of the second absorbent layer; and
wherein the superabsorbent immobilizer consists essentially of one or more polymers having a peak molecular weight of at least about 10 kg/mol, as determined using the gel permeation chromatography method described herein.

19. An absorbent article comprising an absorbent core;
wherein the absorbent core comprises first and second absorbent layers, the first absorbent layer comprising a first substrate and the second absorbent layer comprising a second substrate;
wherein the first and second absorbent layers further comprise superabsorbent polymer material deposited on said first and second substrates and a superabsorbent immobilizer covering the superabsorbent polymer material on the respective first and second substrates;
wherein said first and second absorbent layers are combined together such that at least a portion of the superabsorbent immobilizer of the first absorbent layer contacts at least a portion of the superabsorbent immobilizer of the second absorbent layer; and
wherein the superabsorbent immobilizer is substantially tackifier-free.

20. An absorbent article comprising an absorbent core;
wherein the absorbent core comprises first and second absorbent layers, the first absorbent layer comprising a first substrate and the second absorbent layer comprising a second substrate;
wherein the first and second absorbent layers further comprise superabsorbent polymer material deposited on said first and second substrates and a superabsorbent immobilizer covering the superabsorbent polymer material on the respective first and second substrates;
wherein said first and second absorbent layers are combined together such that at least a portion of the superabsorbent immobilizer of the first absorbent layer contacts at least a portion of the superabsorbent immobilizer of the second absorbent layer; and
wherein the superabsorbent immobilizer comprises at least one polymer having a peak molecular weight of at least about 10 kg/mol, as determined using the gel permeation chromatography method described herein, and wherein the superabsorbent immobilizer is substantially free of any tackifiers, waxes, oils, and plasticizers.
